(19) United States
(12) Patent Application Publication
(10) Pub. No.: US 2018/0294055 A1
(43) Pub. Date: Oct. 11, 2018

(54) AUTOMATED SYSTEM AND METHOD FOR BLOOD SAFETY WORKFLOW VERIFICATION AND VALIDATION

(71) Applicant: Noha Hazzazi, Fairfax, VA (US)

(72) Inventor: Noha Hazzazi, Fairfax, VA (US)

(21) Appl. No.: 16/004,773

(22) Filed: Jun. 11, 2018

Related U.S. Application Data
(60) Provisional application No. 62/517,935, filed on Jun. 11, 2017.

(51) Int. Cl. G16H 40/20; G06Q 10/06; G16H 10/60

(52) U.S. Cl. CPC G16H 40/20; G16H 10/60; G06Q 10/0633

(57) ABSTRACT

A method of using temporal logic technique to verify and validate blood safety workflows, the method includes creating a model workflow of blood supply chain process, wherein humans and machines are included as workflow constructs where involved, translating regulations governing blood safety into statements in temporal logic formula, wherein components satisfying a temporal logic formula correspond to satisfying the translated regulation, combining the translated regulations with the created blood supply chain model workflow, and validating that all translated regulations have been satisfied using a theorem prover.

### BACKGROUND OF THE INVENTION

### 1. Field of the Invention

The present general inventive concept relates to systems and methods of verifying and validating blood safety workflows, and more particularly, to automating the verification and validation of blood safety workflows using temporal logic.

### 2. Background of the Invention

Each year, millions of units of blood is collected and transfused by blood centers and hospitals throughout the United States and the entire World. As such, keeping the blood supply safe is critical to the health and safety of people around the world. In the United States, all of the blood collection facilities' collection and management procedures are guided by strict regulations in order to ensure the safety of the entire blood supply.

That is, in the United States, the Federal Drug Enforcement Administration (FDA), American Association of Blood Banks (AABB) and the College of American Pathologists define standards and regulations and oversee the operations of all the blood banks in the U.S.

The process of managing human blood is governed by federal agencies using stringent safety requirements that span the whole blood supply chain. Compliance to these strict and complicated safety requirements, however, is currently only checked and validated by using periodic audits and validations that are conducted by a human. Understandably, these critical validations and audits are labor intensive and require a considerable amount of time and resources but remain prone to human error. In addition, these validation and audit processes are subject to change, due to constantly evolving technology.

Therefore, what is needed is an automated or semi-automated system that can either automate or assist the labor-intensive checking process and thereby reduce human error in the validation process by installing a machine-based verification process. In addition, what is also needed is a flexible system that can easily adapt to changing standards and regulations and new technology. Further, what is needed is an automated system that is configured to employ logical algorithms to track specific steps within the entire process of procuring, storing, dispensing, and transfusing blood.

### BRIEF SUMMARY OF THE INVENTION

The present general inventive concept provides a verification system and method for ensuring the safety of processes spanning the range from donation to transfusion that contribute to the safety of the vein-to-vein blood supply chain. Exemplary embodiments of the method according to the present general inventive concept includes a semi-automated verification of the processes used in individual steps and their choreography to existing validation processes.

The method begins with registering a donor into the system, checking if the donor is suitable, and collecting units of blood from the donor for a specific purpose (i.e. whole blood vs. apheresis).

The present general inventive concept further includes a method to cover the safety of the remainder of the blood supply chain, including a verification process against standards and regulated requirements defined by government agencies or the like in the laboratory (e.g., unit and sample processing), storage, post donation, and transfusion.

The present general inventive concept provides a novel model of the entire blood supply chain as a workflow, wherein each step is modeled as a process carried out by humans or machines and their choreography is modeled as workflow constructs.

Features and/or utilities of the present general inventive concept may be achieved by providing a method of using temporal logic technique to verify and validate blood safety workflows, the method includes creating a model workflow of blood supply chain process, wherein humans and machines are included as workflow constructs where involved, translating regulations governing blood safety into statements in temporal logic formula, wherein components satisfying a temporal logic formula correspond to satisfying the translated regulation, combining the translated regulations with the created blood supply chain model workflow, and validating that all translated regulations have been satisfied using a theorem prover.

The method may further include a model checker to prove that the created model workflow satisfies all of the regulations governing blood safety.

The method may further include using formal workflow specification and execution environment and language in Yet Another Workflow Model (YAWL) to create the model workflow of the blood supply chain process.

The method may further include using YAWL to check that the created model workflow satisfies reachability, structure, and soundness.

The created model workflow of the blood supply chain process may include registering a donor, conducting a physical exam, conducting an interview, determining eligibility, creating labels, drawing blood, determining post donation status, and transfusing blood.

The validating of the translated regulations may occur automatically.

Additional aspects of the present general inventive concept will be set forth in part in the description which follows and, in part, will be obvious from the description, or may be learned by practice of the general inventive concept.

### BRIEF DESCRIPTIONS OF THE DRAWINGS

These and/or other aspects of the present general inventive concept will become apparent and more readily appreciated from the following description of the embodiments, taken in conjunction with the accompanying drawings of which:

FIG. 1 is a workflow illustrating blood bank processes;

FIG. 2 is a workflow model of an automated verification and validation system according to an exemplary embodiment of the present general inventive concept;

FIG. 3 is a sub-workflow model of a registration process according to an exemplary embodiment of the present general inventive concept;

FIG. 4 is a sub-workflow model of a suitability process according to an exemplary embodiment of the present general inventive concept;

FIG. 5 is a sub-workflow model of a collection process according to an exemplary embodiment of the present general inventive concept;

FIG. 6 is a sub-workflow model of a post-donation process according to an exemplary embodiment of the present general inventive concept;

FIG. 7 is a sub-workflow model of a BTSLAB process according to an exemplary embodiment of the present general inventive concept;

FIG. 8 is a sub-workflow model of a storing unit process according to an exemplary embodiment of the present general inventive concept;

FIG. 9 is a sub-workflow model of a registration deferral process according to an exemplary embodiment of the present general inventive concept;

FIG. 10 is a sub-workflow model of a discarding process according to an exemplary embodiment of the present general inventive concept;

FIG. 11 is a sub-workflow model of a transfusion request process according to an exemplary embodiment of the present general inventive concept;

FIG. 12 is a sub-workflow model of a transfusion to patient process according to an exemplary embodiment of the present general inventive concept;

FIG. 13 is a sub-workflow model of a post transfusion process according to an exemplary embodiment of the present general inventive concept;

FIG. 14 is a sub-workflow model of another registration process (C2) according to an exemplary embodiment of the present general inventive concept;

FIG. 15 is a sub-workflow model of a serology process according to an exemplary embodiment of the present general inventive concept;

FIG. 16 is a sub-workflow model of a unit cross-match process according to an exemplary embodiment of the present general inventive concept;

FIG. 17 is a sub-workflow model of a checking donor ABO process according to an exemplary embodiment of the present general inventive concept;

FIG. 18 is a sub-workflow model of a checking ABO from sample process according to an exemplary embodiment of the present general inventive concept;

FIG. 19 is a sub-workflow model of a checking ABO from segment process according to an exemplary embodiment of the present general inventive concept;

FIG. 20 illustrates a syntax (atomic predicate symbols) to be used throughout the specification and workflow diagrams;

FIGS. 21 and 22 illustrate a summary of commonly used semantics of temporal logic;

FIG. 23 illustrates workflows modelled as state transitions between so-called states of the blood supply chain;

FIG. 24 illustrates registration workflow modelled as state transitions between so-called states of the blood supply chain;

FIG. 25 illustrates suitability workflow modelled as state transitions between so-called states of the blood supply chain;

FIG. 26 illustrates collection workflow modelled as state transitions between so-called states of the blood supply chain;

FIG. 27 illustrates post donation workflow modelled as state transitions between so-called states of the blood supply chain;

FIG. 28 illustrates BTSLAB workflow modelled as state transitions between so-called states of the blood supply chain;

FIG. 29 illustrates unit processing workflow modelled as state transitions between so-called states of the blood supply chain;

FIG. 30 illustrates sample processing workflow modelled as state transitions between so-called states of the blood supply chain;

FIG. 31 illustrates serology workflow modelled as state transitions between so-called states of the blood supply chain;

FIG. 32 illustrates ABO workflow modelled as state transitions between so-called states of the blood supply chain;

FIG. 33 illustrates ABO from sample workflow modelled as state transitions between so-called states of the blood supply chain;

FIG. 34 illustrates ABO from segment workflow modelled as state transitions between so-called states of the blood supply chain;

FIG. 35 illustrates transfusion request workflow modelled as state transitions between so-called states of the blood supply chain;

FIG. 36 illustrates cross-match workflow modelled as state transitions between so-called states of the blood supply chain;

FIG. 37 illustrates transfusion workflow modelled as state transitions between so-called states of the blood supply chain;

FIG. 38 illustrates patient ABO workflow modelled as state transitions between so-called states of the blood supply chain;

FIG. 39 illustrates post transfusion workflow modelled as state transitions between so-called states of the blood supply chain;

FIG. 40 illustrates adverse reaction type workflow modelled as state transitions between so-called states of the blood supply chain;

FIG. 41 illustrates acute reaction workflow modelled as state transitions between so-called states of the blood supply chain;

FIG. 42 illustrates delayed reaction workflow modelled as state transitions between so-called states of the blood supply chain;

FIG. 43 illustrates Lemmas Structure; and

FIG. 44 illustrates a flow chart of the modelled workflow illustrated in FIG. 23.

### DETAILED DESCRIPTION

The present general inventive concept includes the disclosure provided within the paper titled Automating the Verification of Blood Safety Workflow by Noha Hazzazi and is hereby incorporated herein in its entirety by reference.

Blood transfusion is a common procedure used all over the world. Due to safety related issues such as blood quality, contamination, aging, etc. The United States and other countries have placed strict regulations and standards in place to ensure that the blood delivered for transfusion is safe for donors and recipients. Between donation and transfusion, blood products go through many steps in a supply chain. Decomposing donated blood into commonly transfused components, testing them against disease agents and storing them to satisfy safety of the recipient are some standard steps of this supply chain. Thus, the ultimate safety of the transfused blood depends on being safe at every step of the supply chain, most of which are governed by regulation. Traditionally the safety at each stage is validated by performing regulated checks and by having an inspection process to ensure regulatory compliance of these processes themselves.

The present general inventive concept provides a verification system and method for ensuring the safety of processes spanning the range from donation to transfusion that contribute to the safety of the vein-to-vein blood supply chain. Exemplary embodiments of the method according to the present general inventive concept includes a semi-automated verification of the processes used in individual steps and their choreography to existing validation processes.

The method begins with registering a donor into the system, checking if the donor is suitable, and collecting units of blood from the donor for a specific purpose (i.e. whole blood vs. apheresis).

The present general inventive concept further includes a method to cover the safety of the remainder of the blood supply chain, including a verification process against standards and regulated requirements defined by government agencies or the like in the laboratory (e.g., unit and sample processing), storage, post donation, and transfusion.

The present general inventive concept provides a novel model of the entire blood supply chain as a workflow, wherein each step is modeled as a process carried out by humans or machines and their choreography is modeled as workflow constructs.

The system and method initially define all of the safety regulations and standards specified by all regulation bodies including the FDA, the AABB, the CAP and then associates relevant regulations that should be satisfied by appropriate components of the modeled workflow.

The system and method then translates the regulations to statements in Temporal Logic, thereby creating a workflow model where appropriate components satisfying a temporal logic formula (that formally states the safety condition) should ensure that the blood supply chain complies with the mandated safety regulations.

The system and method further include an automated translator configured to translate the workflows and the temporal logic formulas attached to appropriate fragments of the workflow and then use a theorem prover in order to validate that all of the mandated safety conditions are satisfied by the blood processing supply chain.

A workflow is a term that describes tasks, procedural steps, routines and sub-routines, input data, output data, and resources that is used by a particular process. A workflow may be used to represent one or more processes which may be linked together by a plurality of rules.

Reference will now be made in detail to the exemplary embodiments of the present general inventive concept, examples of which are illustrated in the accompanying drawings, wherein like reference numerals refer to the like elements throughout. The exemplary embodiments are described below in order to explain the present general inventive concept by referring to the FIGS.

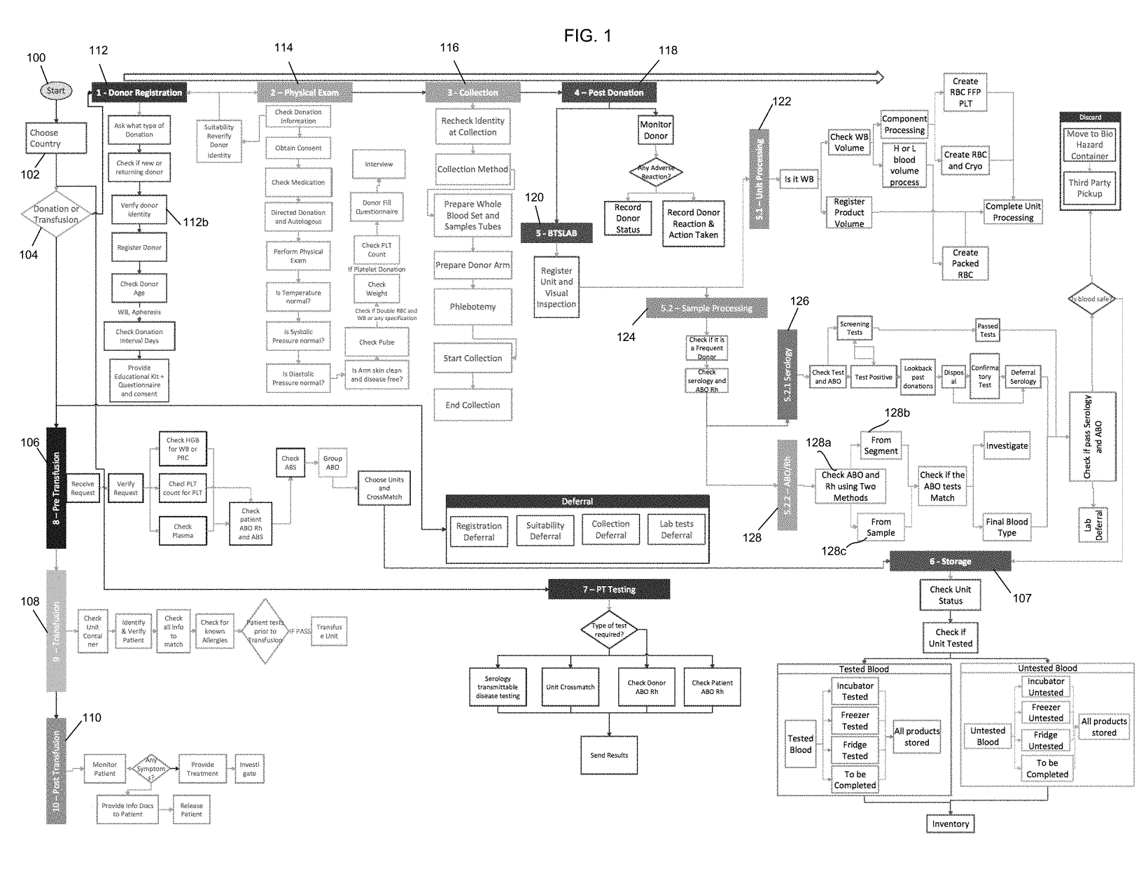

FIG. 1 is a workflow illustrating blood bank processes. Referring to FIG. 1, according to exemplary embodiments of the present general inventive concept, the automated verification and validation system provides a complete translation of all relevant regulations governing blood safety into statements in temporal logic formula to create a workflow model. The automated verification and validation system further includes an automated translator to translate the modelled workflows and the temporal logic formulas and a theorem prover to validate that all of the safety conditions have been satisfied.

In exemplary embodiments, a method of using temporal logic technique to verify and validate blood safety workflows includes creating a model workflow of blood supply chain process, wherein humans and machines are included as workflow constructs where involved, translating regulations governing blood safety into statements in temporal logic formula, wherein components satisfying a temporal logic formula correspond to satisfying the translated regulation, combining the translated regulations with the created blood supply chain model workflow, and validating that all translated regulations have been satisfied using a theorem prover.

Referring to FIG. 1, in the present exemplary embodiment, the blood supply chain process includes a user selecting a specific geographic location to receive a blood supply unit (e.g., country, state, city or town) in order to incorporate the regulations specific to each region into the system (step 102). Next, the user selects whether the blood supply unit is to undergo the donation process, the transfusion process, or the proficiency testing process (step 104), wherein each process is independent as the donation process of collecting the blood products and the transfusion process is the consumer of the stored blood products.

If the user selects transfusion, the system initiates the pre-transfusion process (step 106) which includes receiving a request (step 106a), verifying the request (step 106b), checking HGB for WB or PRC (step 106c), checking PLT count for PLT (step 106d), and checking plasma (step 106e), checking the patient's ABO Rh and ABS (step 106f), checking ABS (step 106g), grouping ABO (step 106h), and then choosing units and crossmatch (step 106i). Finally, the blood supply unit is submitted for storage.

When the blood supply unit is submitted for storage, the system initiates the storage process (step 107) which includes checking unit status (step 107a), checking if unit has been tested (step 107b), and submitting the untested blood unit for storage (step 107c) and the tested blood unit for storage (step 107d). The blood supply unit is then submitted into inventory (step 107e).

The donation process modeled in the workflow illustrated in FIG. 1 identifies, selects, and performs a physical examination for a donor's suitability for a blood donation. In addition, various other details such as hemoglobin level, collection date and time, etc. are taken into account to ensure that the donor is appropriate to donate, and the blood is safe to be processed. In all cases, the donor's record and information are kept confidential, as the donor's identification number is the only shared information used throughout the blood workflow while the donor's personal information is maintained confidential and kept private.

If the user selects donation, the system initiates the donor registration process (step 112) which includes determining the type of donation (step 112a), determining whether the donor is new or a returning donor (step 112b), verifying the identity of the donor (step 112c), registering the donor (step 112d), checking the age of the donor (step 112e), checking the donation interval days (step 112f), and providing educational information and consent to the donor (step 112g). That is, the donation process includes a pre-donation sub-phase, a donation sub-phase, and a post-donation sub-phase. The registration process captures donor demographics prior to the mandated physical exam. This registration process requires that the donor be identified, registered, and provided with educational material, all stemming from a safety measure mandate of tracking all processes from registration until the donor follow-ups.

Next, the system initiates the physical exam process (114) which includes checking donation information (step 114a), obtaining consent (step 114b), checking medication (step 114c), directing donation and autologous (step 114d), performing physical exam on donor (step 114e), checking donor temperature (step 114f), checking systolic pressure of donor (step 114g), checking diastolic pressure of donor (step 114h), determining whether donor's skin is clean and disease free (step 114i), checking donors pulse (step 114j), checking weight of donor (step 114k), checking PLT count (step 114l), receiving questionnaire from donor (step 114m), and interviewing the donor (step 114n).

Next, the system initiates the collection process (step 116) which includes rechecking identity of the donor (step 116a), determining the collection method (step 116b), preparing whole blood set and samples tubes (step 116c), preparing donor arm for collection (step 116d), performing phlebotomy (step 116e), starting collection of the blood (step 116f), and ending collection of the blood (step 116g).

Next, the system initiates the post-donation process (step 118) which includes monitoring the donor (step 118a), determining whether there are any adverse reactions (step 118b), recording the donor status (118c), and then recording the donor reaction and the action taken (step 118d).

Next, the system initiates the BTSLAB process (step 120) which includes unit processing (step 122) and sample processing (step 124). The BTSLAB process (step 120) includes inspecting and registering the blood unit (step 120a).

The unit processing process (step 122) includes determining whether the blood unit is WB (step 122a), checking WB volume (step 122b), processing components (step 122c), creating RBC PLT (step 122d), creating RBC and cyro (step 122e), selecting between H or L blood volume process (step 122f), creating packed RBC (step 122g), and completing unit processing (step 122h).

The sample processing (step 124) includes determining whether the donor is a frequent donor (step 124a) and checking serology and ABP Rh (step 124b). Next, the sample processing (step 124) further includes serology process (step 126) and ABO/Rh process (step 128).

The serology process (step 126) includes checking test and ABO (step 126a) of the blood unit, performing screening tests (step 126b) on the blood unit, if the blood unit passed and is determined safe, it is sent to storage (step 107). Otherwise, the blood unit is sent to biohazard container (step 109).

The ABO/Rh process (step 128) includes checking ABO and Rh using a plurality of methods (step 128a) (i) from segment (step 128b) and from sample (step 128c). The ABO/Rh process further includes determining whether the ABO tests match from the plurality of methods used (step 128d), performing investigations (step 128e) and determining final blood type (step 128f). Finally, if the blood unit passed and is determined safe, it is sent to storage (step 107). Otherwise, the blood unit is sent to biohazard container (step 109).

Blood transfusions have been used to save many lives over the years. The process of blood transfusion begins with a donation and ends with transfusing blood products to one or more patients. In between the blood draw and transfusion, blood products go through many tests and procedures. These procedures can be categorized into phases (such as pre-analytical, analytical and post-analytical). Every phase consists of many tasks that are required to follow specific regulations and standards. These regulations have been specified by the US Food and Drug Administration (FDA), AABB formerly known as American Association of Blood Bank and the College of American Pathologist (CAP).

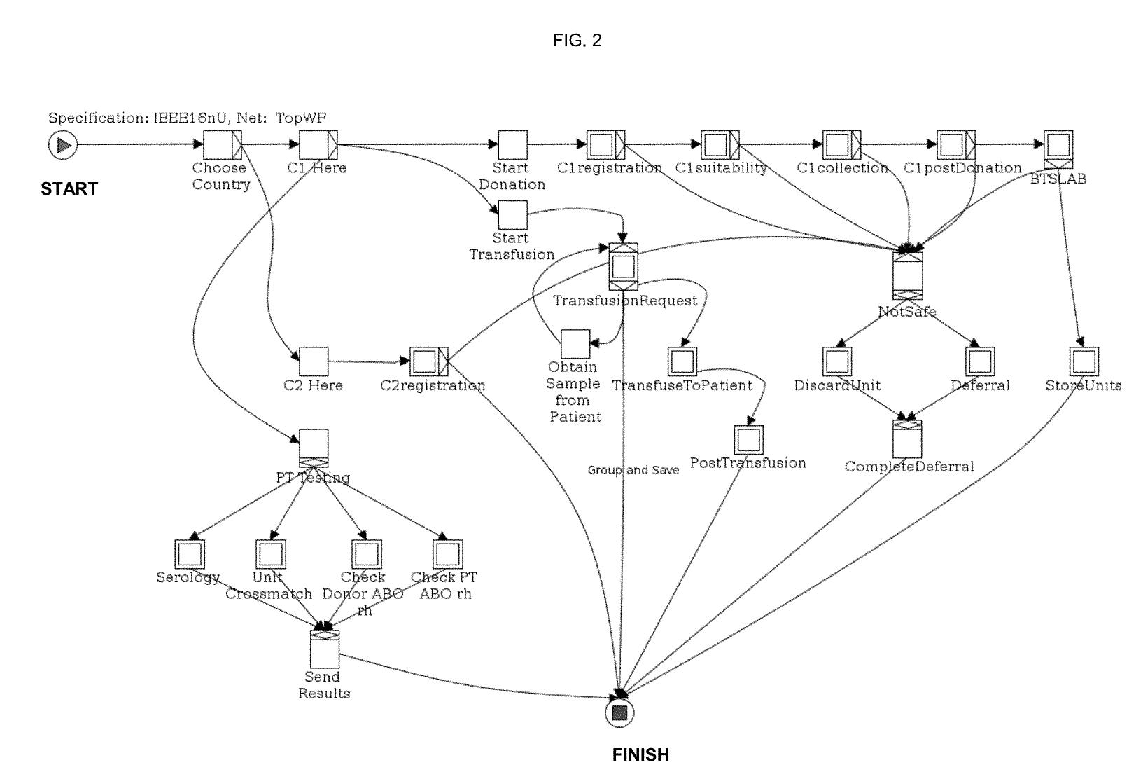

FIG. 2 is a workflow model of an automated verification and validation system according to an exemplary embodiment of the present general inventive concept.

FIG. 2 illustrates high-level main workflows consisting of multiple sub-workflow tasks. Each task in the workflow is dependent on the execution of the previous task with expected results, thereby creating the blood supply chain. The workflow according to the present general inventive concept covers pre-analytical, analytical and post-analytical phases in each of the processes of donation, laboratory, and transfusion. Processes in the Pre-analytical phase are called prior to blood collection, transfusion, and testing. These processes check donor's suitability criteria to donate blood, blood labeling laboratory sample and unit handling and checking the transfusion request form. Processes in the analytical phase perform the testing, donation, and transfusion. Processes in the Postanalytical phase creates the final report of tests, monitor the donor and/or recipient.

The workflow according to the present general inventive concept further includes safety requirements based various regulations, including the Food and Drug Administration (FDA), the American Association of Blood Bank (AABB), and the College of America Pathologist (CAP) specifications, in each of the atomic and complex tasks. This specification refers to the specified requirements from CAP notation as (TRM.<number>), FDA as (CFR <number>), AABB as (standard <number>). To describe the entire process, FIG. 1 illustrates high-level blood bank processes. The initial process is to identify the country then followed by a choice of the next process. The next process can be the donation process or the transfusion process or Proficiency Testing (PT) process. Each of the processes is independent as the donation process is the collector of the blood products and the transfusion process is the consumer of the stored blood products.

### Donation Process

The safety of the blood workflow starts with the donation process in the pre-donation phase. The donation process consists of pre-donation sub-phase, donation sub-phase, and post-donation sub-phase. The donation process is important as donated blood affects the blood products transfused to recipients. The donation process modeled in the workflow identify, select and perform the physical examination for donor's suitability for the donation shown in FIG. 1. Other details such as hemoglobin level, collection date and time, etc. are taken into account to ensure that the donor is appropriate to donate, and the blood is safe to be processed (TRM.45252). Donor's record and information are kept confidential, as the donation ID is the only shared information used throughout the blood workflow while the donor's demographics are not shared (TRM.45253).

### Pre-Donation

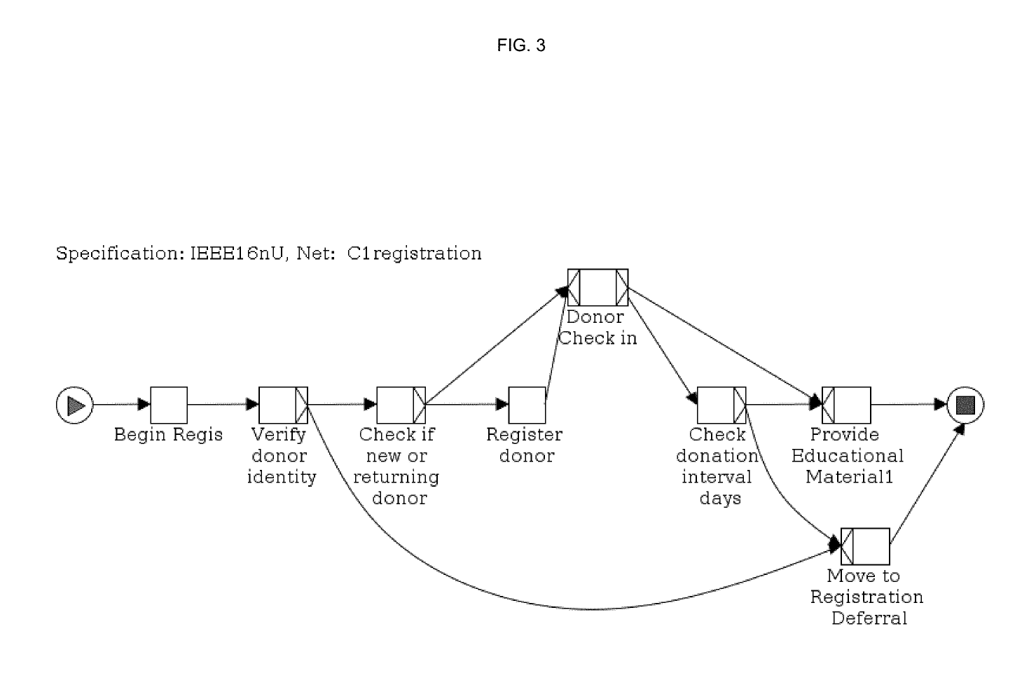

FIG. 3 is a sub-workflow model of a registration process according to an exemplary embodiment of the present general inventive concept.

Referring now to FIGS. 1, 2, and 3, the first main process of blood bank operations is Registration step 112 that captures donor demographics prior to the mandated Physical Exam step 114. The process according to the present invention and specification requires the donor to be identified, registered, and provided with educational material, all stemming from a safety measure mandate of tracking all processes from registration until the patient follow-ups. For example, the detailed procedure in the Identify & Verify Donor step 112b involves identifying the donor by matching his/her photo ID with the person, verifying the ID expiration date and validity. The system then checks donors' suitability based on the standard for donor demographics (e.g., at least of age 17 for allogeneic donation and parental consent is required if underage; CAP TRM.45256). In addition, the inter-donation time interval of the donor is checked depending on the type of donation, past donation and amount donated to ensure that the donor's safety is ensured after donating blood this time.

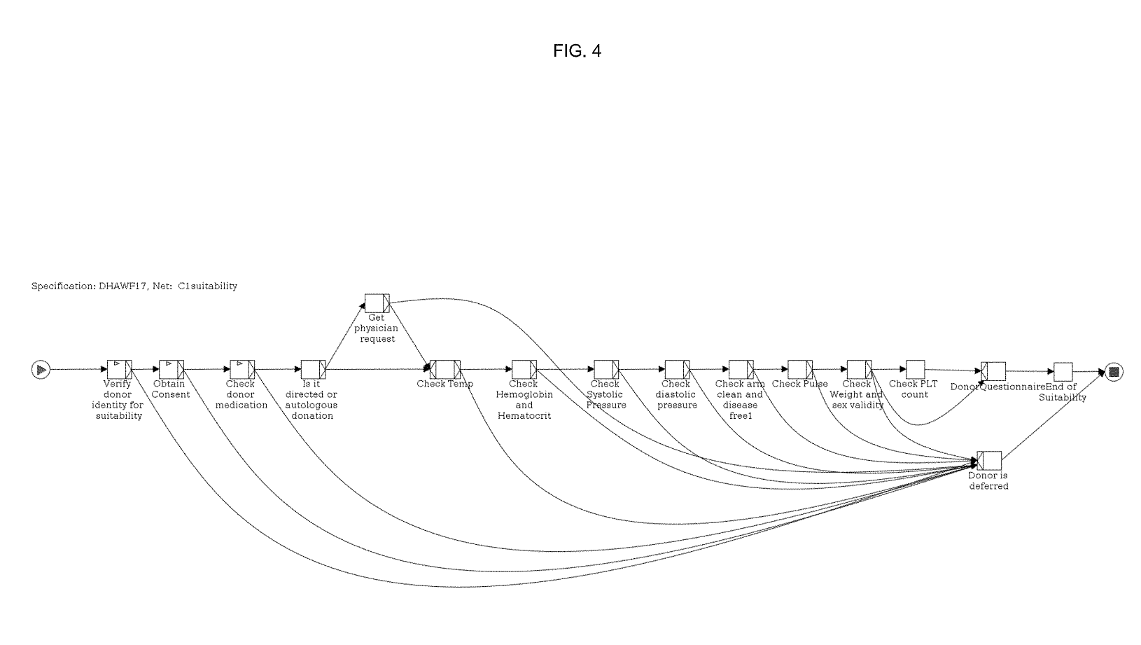

FIG. 4 is a sub-workflow model of a suitability process according to an exemplary embodiment of the present general inventive concept.

Referring now to FIGS. 1, 2, and 4, the next main process is the Physical Exam step 114 that determines the donor's suitability for donating blood. This composite process depends on the type of donation, as there are separate regulatory requirements for whole blood or apheresis donations. As a component of the physical exam, the donor is also required to provide his/her consent and pass a relevant health interview (CAP TRM.42222, 45263, 45257, 45258, 45259, 45261, AABB standard part 5.2.3). The suitability process checks also the type of donation for specific requirements such as a physician's note before autologous donation (CAP TRM.45270, 45271). The workflow model used with the present general inventive concept is a chain of processes that is capable of logging data throughout the blood bank supply chain. These processes allow a user or operator to retrieve records of each step throughout the blood bank supply chain for auditing and tracking (CAP TRM.42223, 47320). The hemoglobin level, blood pressure, temperature, arm diseases, and the inter-donation interval are checked for whole blood donors. These measures are specified by the FDA to ensure the safety of the donor and the donated blood sample. For example, FDA's CFR 640.3 specifies that (1) normal temperature and (2) systolic and diastolic blood pressures are within normal limits. However, no quantifiable definition of normal is provided. For this reason, many blood banks apply other standards (e.g. AABB) in support of the FDA's specification. For example, the AABB defines thresholds of ! 37.5° C. for temperature, ! 180 mmHg for Systolic pressure, and ! 100 mm Hg for Diastolic pressure. Regarding the other requirements for whole blood, FDA specifies that a donor must (a) have a hemoglobin level “12.5 grams for allogeneic donations and “11 grams for autologous donations, (b) be free from skin diseases or arm scars, and (c) from diseases transmissible by blood. The CFR leaves the (4) normal pulse rate open, but the AABB Technical Manual states that the pulse rate should be between “50 and ! 100 beats per minute. This additional parameter may be used for the pulse rate. In addition, the AABB has defined specific medications and the last duration and dosage taken to ensure donor's and donated blood's safety. Donor's weight is another requirement AABB has specified to be greater than or equal to 110 pounds for most donations. An apheresis donor eligibility is also checked by identifying donation interval if previously donated and completing a standard questionnaire regarding suitability requirements prior to donating (CAP TRM.42214, 42215, 42220, 42222, 42240, 45276, FDA CFR 640.3, CFR 640.63, CFR 610.40, AABB standard part 5.4.1A). Apheresis requirements for donor suitability differ depending on the type of donation. There are several apheresis procedures such as Plasma Apheresis, Platelet (PLT) apheresis, Double RBC Apheresis, etc. All donations follow the same physical exam requirement as whole blood in most cases. However, donors are checked to ensure that their total serum protein is greater than or equal to 6.0 grams per milliliters of blood and their weight is greater than or equal to 110 pounds (in CFR640.63, section C5). Although the FDA CFR 640.63 specified these requirements, Plateletpheresis will require the donor to have a minimum of 150000 platelets per microliter based on the AABB standard to control safety. Double RBC donations will require male donors height to be “5′1″ and weight “130 and female donors height to be “5′5″ and weight “150. We modeled the physical exam/suitability process based on these requirements.

### Donation

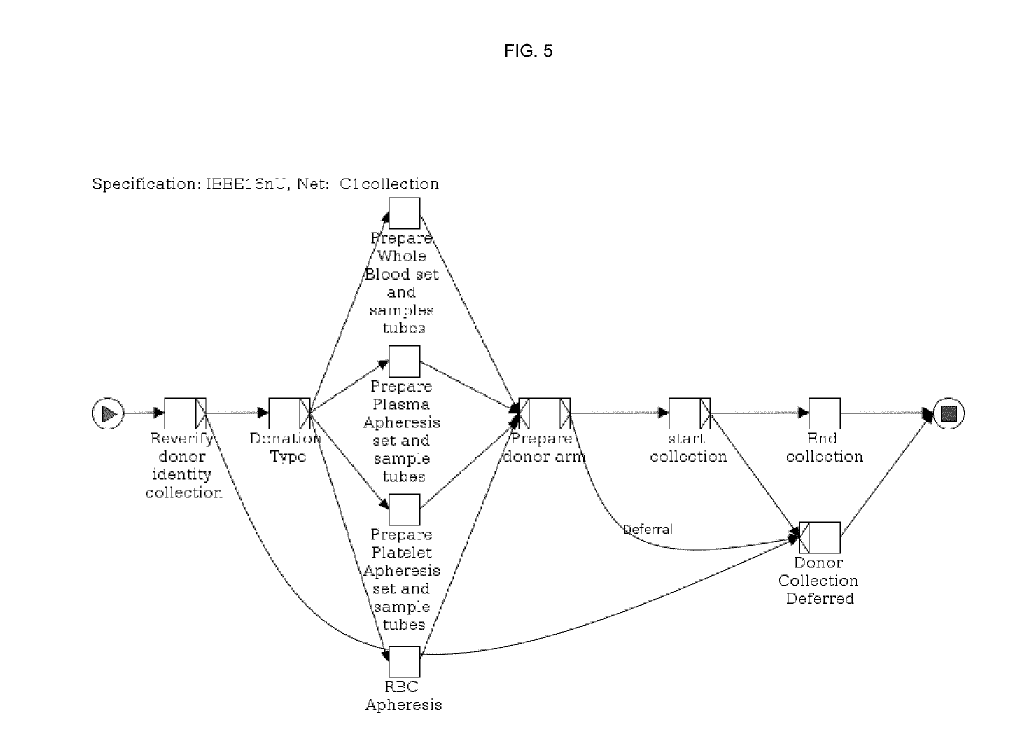

FIG. 5 is a sub-workflow model of a collection process according to an exemplary embodiment of the present general inventive concept.

Referring now to FIGS. 1, 2, and 5, once eligibility is established, the donor is ready for the Collection step 116. Collections also have specific requirements for regular donations (whole blood) and apheresis, which covers aspects such as unit identification, donor arm preparation, and unit temperature control (AABB standard parts 5.3.2, 5.6.3.2, in [55]). The next step is to check the donor arm to ensure that there is a clearly visible vein with no signs of needling (CAP TRM.45275). Whole blood and apheresis require each unit to be linked to a donor via his/her donor ID. FDA's CFR 640.4 states that the skin of the donor at the site of phlebotomy shall be prepared thoroughly and carefully by a method that gives maximum assurance of a sterile container of blood and a proper donor arm preparation to prevent blood contamination (CAP TRM.45267). Controlling the temperature of the blood bag after the collection is another requirement that the FDA specify as collected blood to be placed in storage at a temperature between 1° C. and 6° C. or 21° C. and 24° C. immediately after whole blood collections depending on the component preparation. The FDA CFRs 640.4, 640.14, 640.22, and 640.32 provide information about whole blood collection but are not as specific on either apheresis collection or the volume of blood collected. For this reason, the present inventive concept utilizes AABB specification for the volume of blood required on whole blood and apheresis collections (5.6.4 and 5.5.3.5, respectively, in [55]). The donor is monitored for any reactions during collection and once a collection is completed the system sets donation interval time depending on the type of donation collected.

### Post Donation

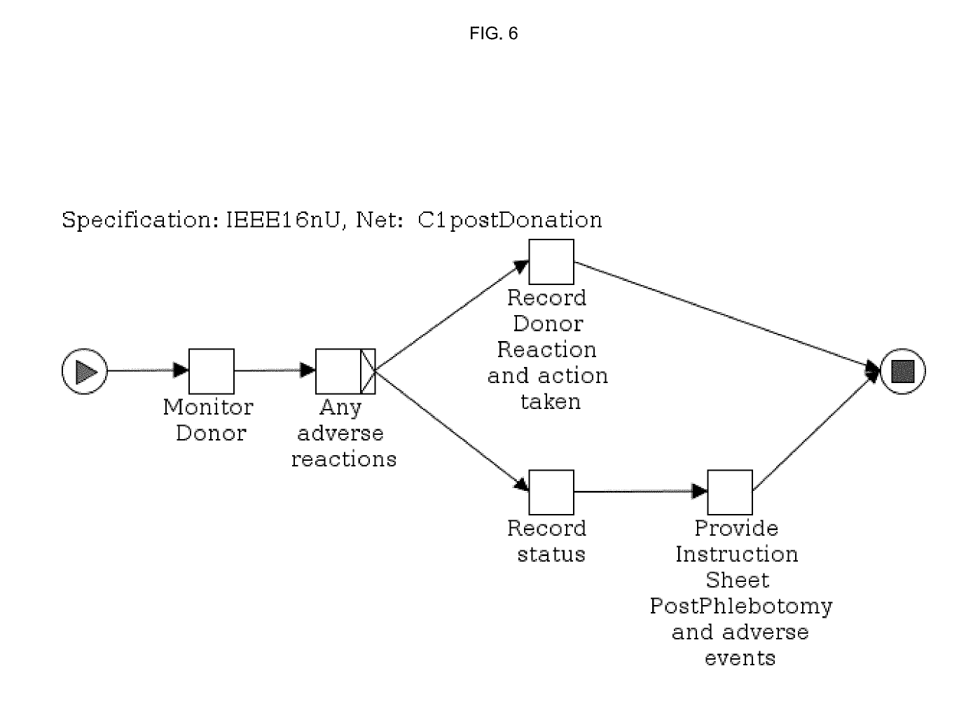

FIG. 6 is a sub-workflow model of a post-donation process according to an exemplary embodiment of the present general inventive concept.

Referring now to FIGS. 1, 2, and 6, the step after collection is post donation step 118, consisting of blood processing and donor monitoring. As stated by blood bank professionals, the donor is monitored to ensure he/she does not develop any adverse reactions to ensure his/her own safety (CAP TRM.45273). It is mandated by the FDA in CFR 640.5 to collect extra samples of the donated blood unit at the time of collection for testing (modeled in FIG. 1). CFR 640.5 states that all laboratory tests shall be made on a specimen of blood taken from the donor at collection time.

### Laboratory Process

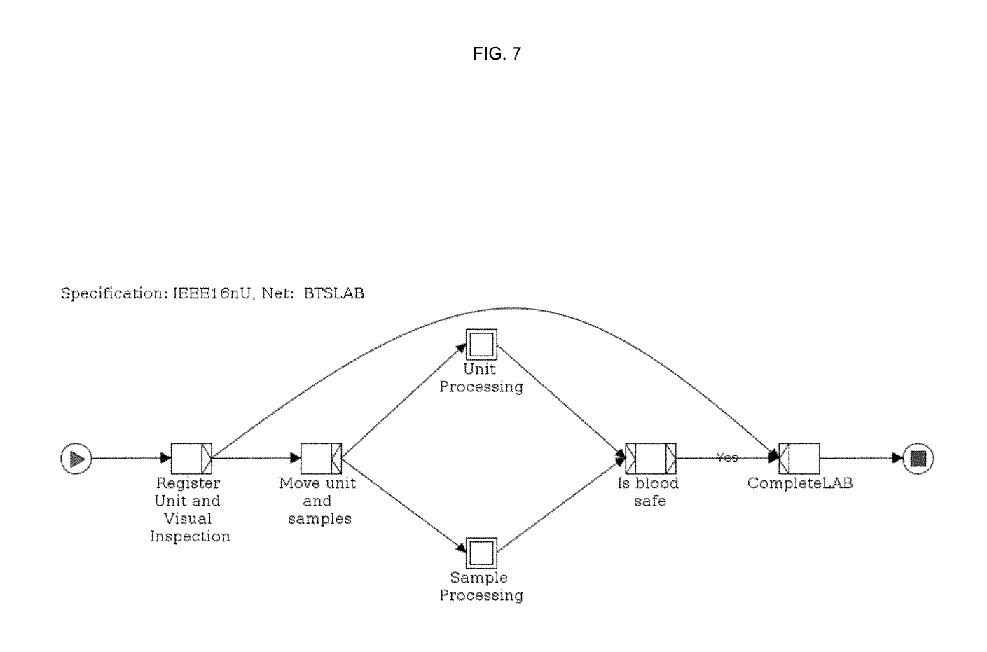

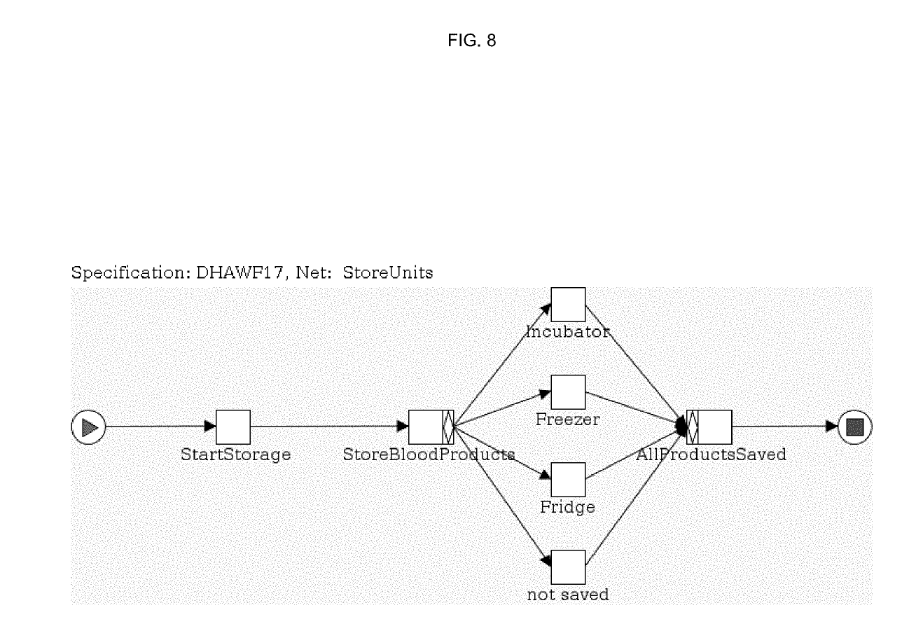

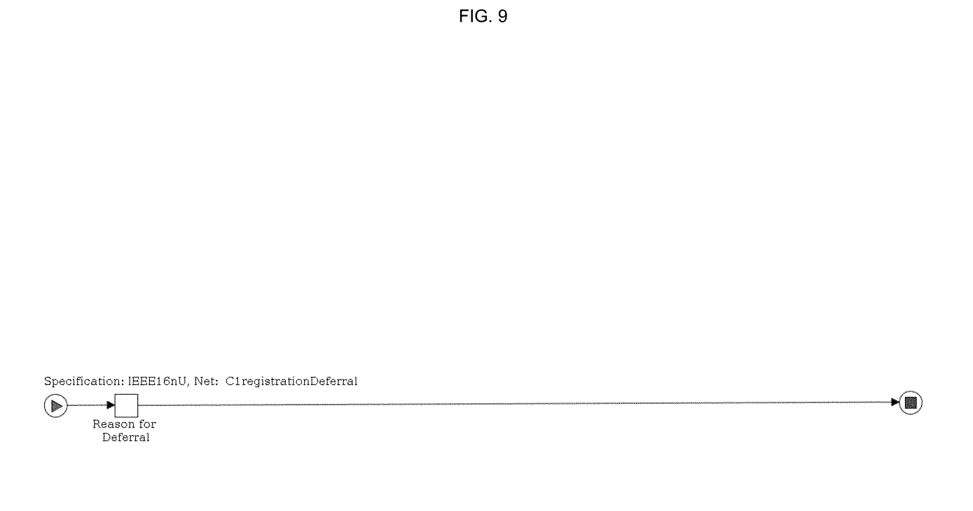

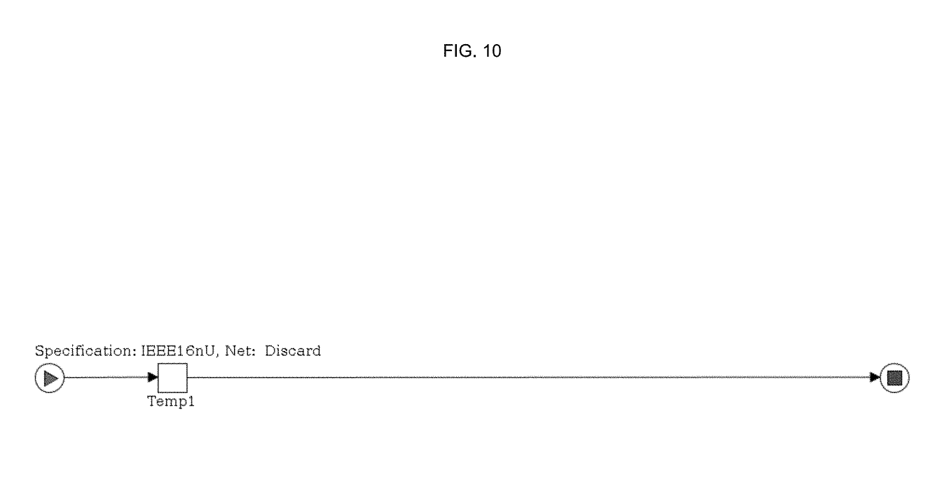

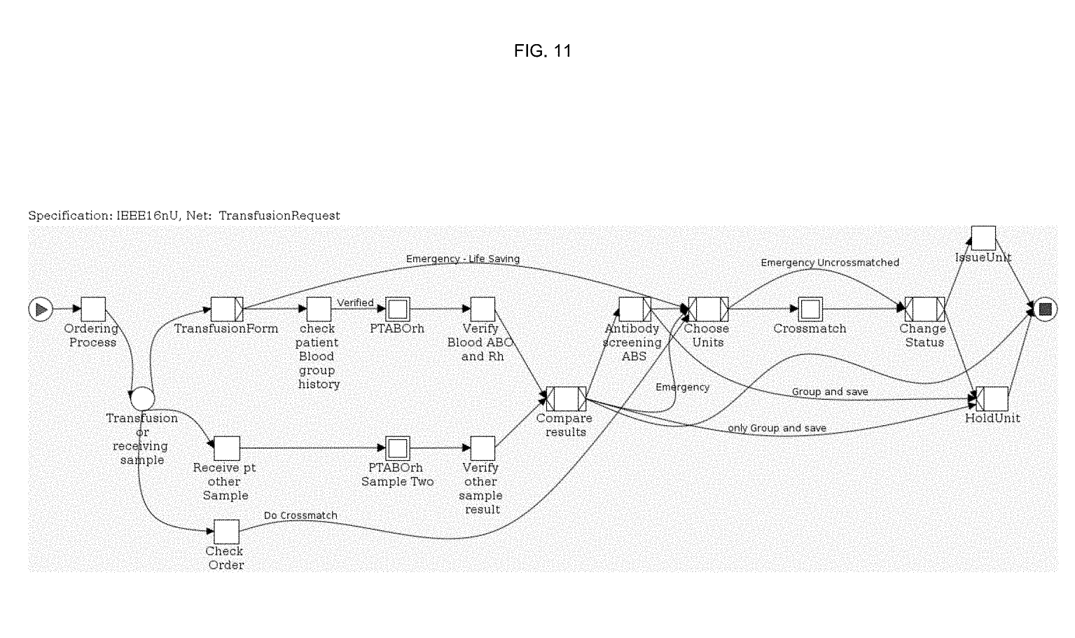

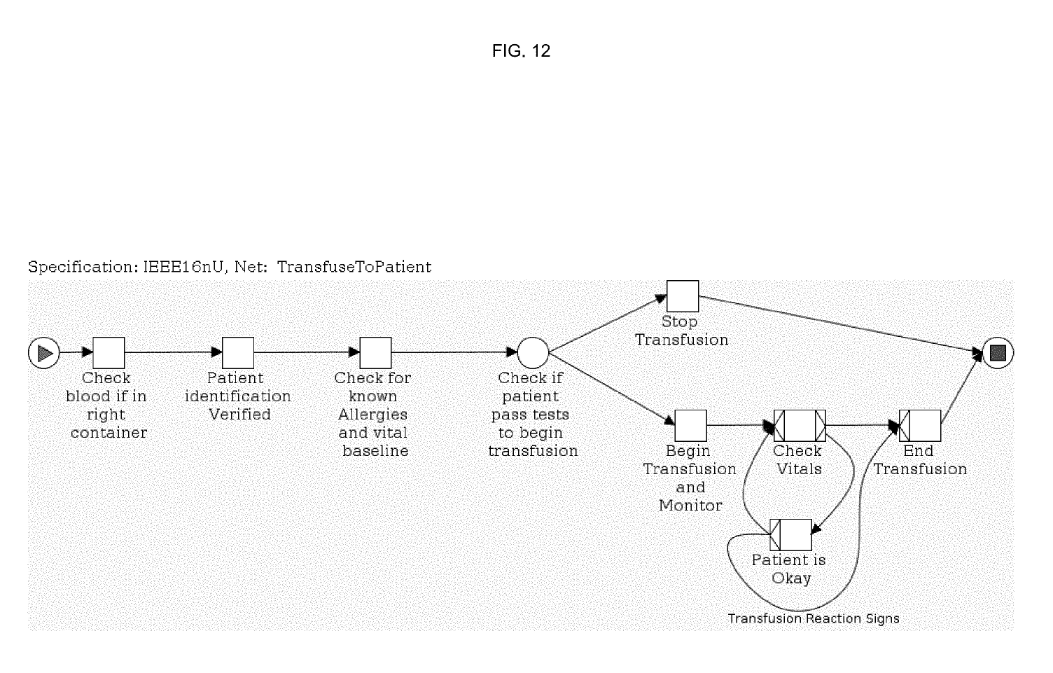

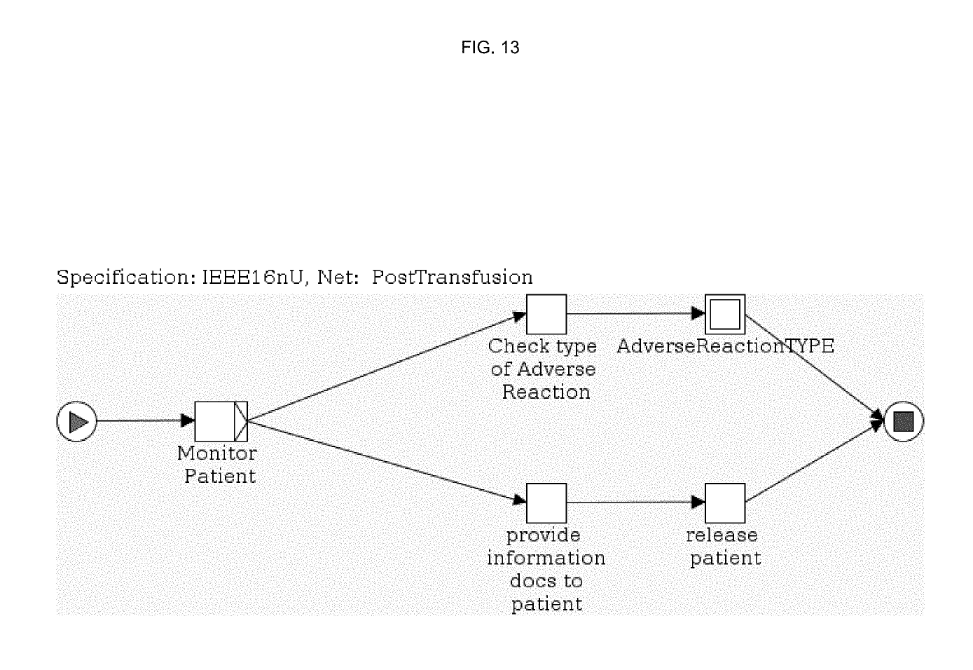

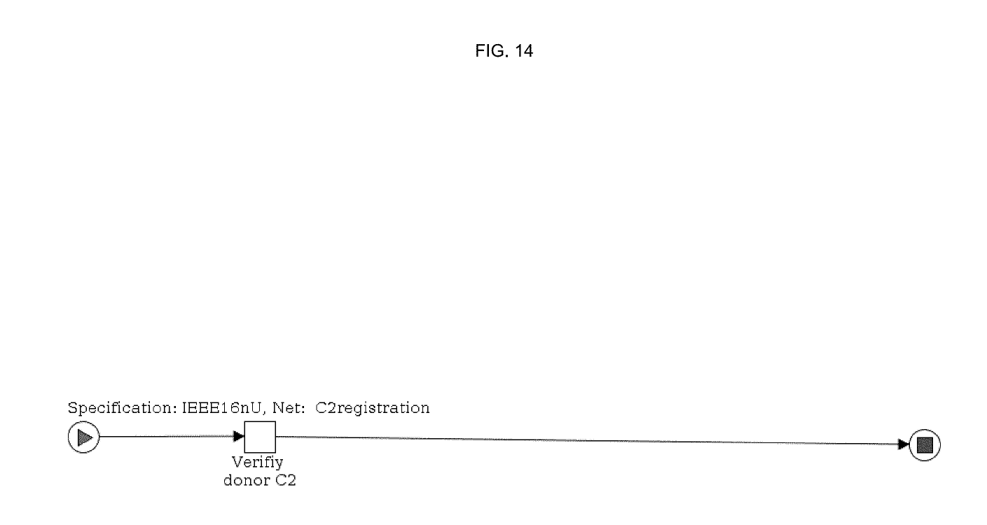

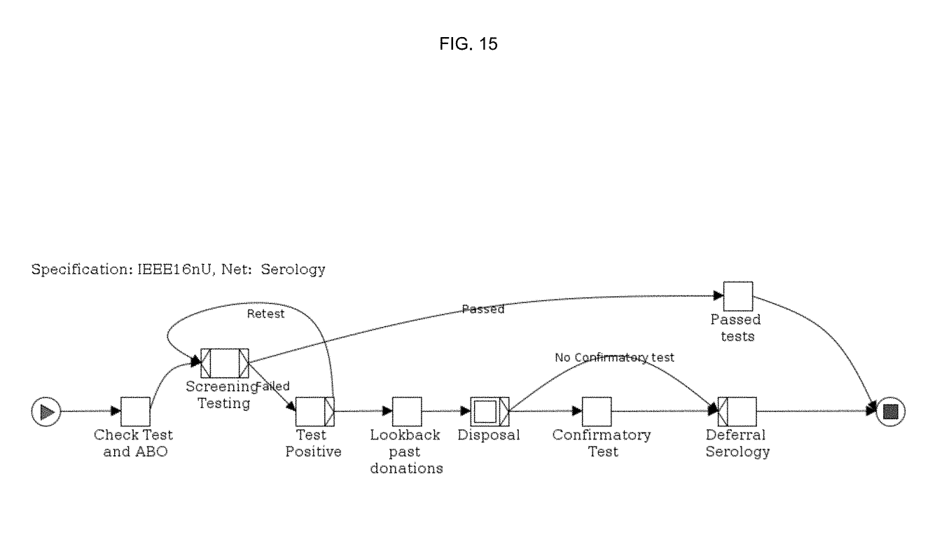

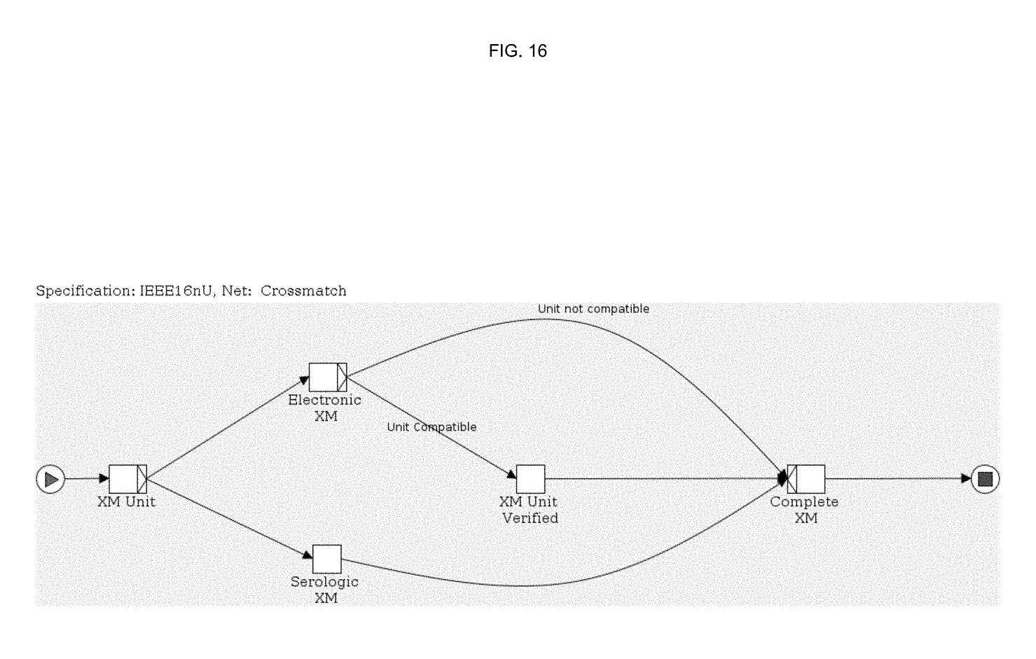

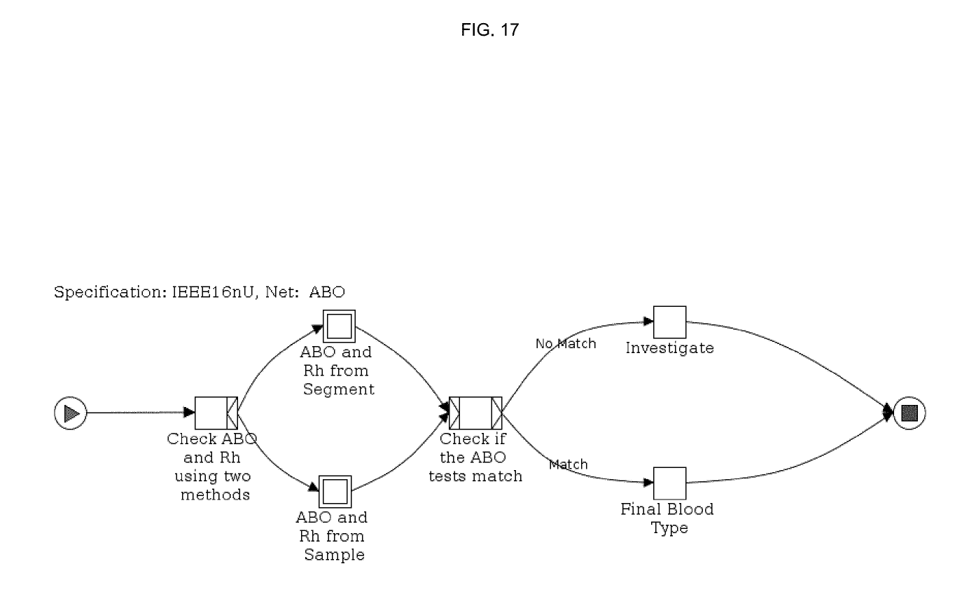

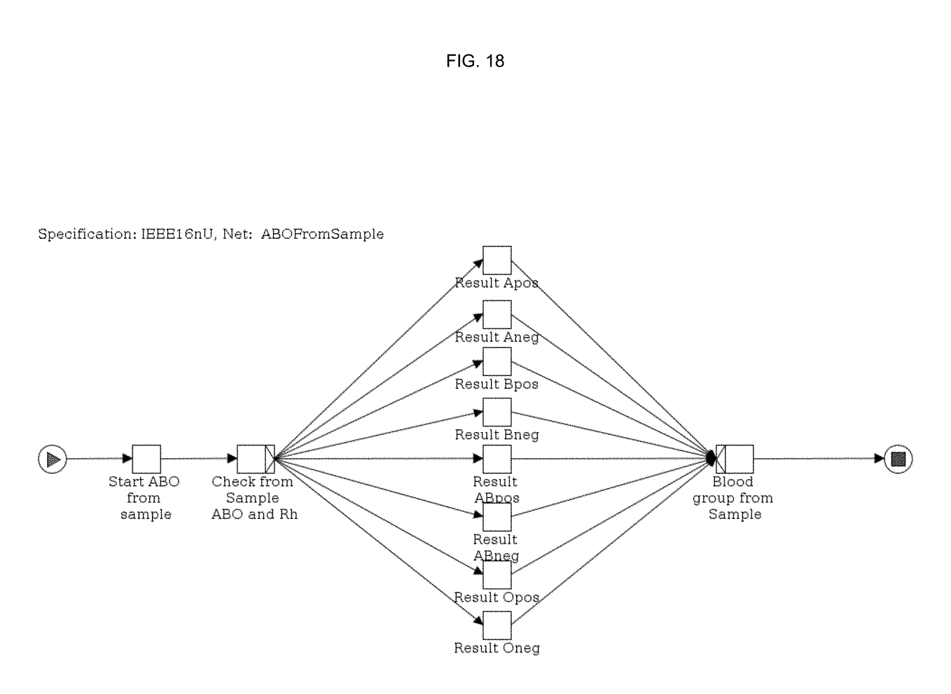

FIG. 7 is a sub-workflow model of a BTSLAB process according to an exemplary embodiment of the present general inventive concept.

Referring to FIGS. 1, 2, and 7, the model of the laboratory process step 120 is shown as BTSLAB. This process is the central component of the blood workflow as the donation process is the producer of the available blood inventory and the transfusion process is the consumer of the blood inventory. One of the main objectives of the laboratory process is to ensure that all tests are preformed correctly and labeled correctly. In this process, blood and blood components are tested and inspected prior to marking the status of an output blood bag's status as available or quarantined. This process consists of the three phases of pre-analytical, analytical and post analytical, explained as follows.

### Processing During Pre-Analytical Stage

The pre-analytical phase starts with the laboratory receiving a unit of blood along with all the data gathered during the blood draw. The unit is visually inspected for leakage and data such as color, time and temperature are entered into the BTSLAB process as shown in FIG. 1 in step 120. The model checks for the regulatory requirements in preparing the unit for testing. However, in situations when the received unit leaks or does not pass the visual inspection, the system directs the lab personnel to dispose of the unit. All donated blood shall be visually inspected and the FDA states that it should be inspected at storage and prior to using. This is shown in FIG. 2 the connection from the BTSLAB to NotSafe process where the unit is disposed of using a third party (CAP TRM.30800). Further tests must be done for sterility, blood unit inspection, and test for communicable disease based on CFR 640.5 (Sections d, e, f). The list of communicable diseases required to check is covered in CFR 610.40 and are included in the model.

### Processing During Analytical Stage

The analytical phase in the laboratory (BTSLAB) is shown in FIG. 2. The Laboratory has two sub-processes that run in parallel shown in FIG. 1, where the unit is processed into final products, such as red blood cells, platelets, plasma etc, while the unit sample undergoes standardized testing for blood grouping, antibody screening and infectious diseases (FDA CFR 640.5, 610.4). All unit information is stored in the system and the units are labeled accordingly. The system checks for compliance with all applicable standards before releasing the unit. Recipient blood sample is also tested in the lab for blood grouping and antibodies. CFR 640.5 also specifies the main tests that are required in processing the blood unit and their acceptable results. These includes the serological test for syphilis, blood grouping, and Rh tests. In FIG. 1, the FDA mandated requirement for the method used for blood grouping was modelled. This is stated in CFR640.5 part (b) as at least two blood group tests shall be made, and the unit shall not be issued until grouping tests by different methods or with different lots of antiserums are in agreement. Regarding Rh grouping, most blood banks currently carry out both the blood grouping and Rh tests in parallel, which have been modelled within the present inventive concept.

The first step after the donation is to take the blood components to the processing laboratory (BTS Lab) and process the bag as shown in step 120 of FIG. 1. This process consists of many interconnected sub-processes such as the Unit Processing and Sample Processing and Discarding. Each of these sub-processes has their own sub-processes as shown below step 120 in steps 122, 124, 126, and 128 illustrated in FIG. 1. Unit Processing (step 122) and Sample Processing (step 124) are executed simultaneously and explained in the subsequent section. Donated whole blood units are separated and processed into different blood product depending on the blood bank setup and the current demand for blood products within the BTS lab, as shown in processes within step 122 of FIG. 1. The BTS lab receives the blood units then are visually inspected and other parameters such as time, temperature, color, and blood volume are checked to ensure that they are within acceptable safety ranges. Units with a lower than standard blood volume are automatically sent to generate packed red blood cells and discard other components. Description of the volume requirements are in the AABB Technical Manual Chapter 6 and AABB standard 5.7.4.7 [3, 56]. Concurrently the FDA has also defined blood processing requirements for each of the blood products such as Whole blood, Red blood cells, Plasma, Platelets and Cryoprecipitate described in CFR 640 [2]. The requirements are to ensure that the received whole blood is in a proper container with the right temperature and set of bags connected to the unit depending on the set of products to be generated.

Referring to FIGS. 1 and 2, sample processing checks for the existence of infectious agents within the blood sample and is shown in step 124. Sample processing consists of multiple sub-processes, dedicating one sub-process for each test. Examples of these processes include ABO Rh and Antibody screening, and serologic and molecular testing for detection of infectious disease agents such as Human T-lymphotropic virus (HTLV), human immunodeficiency virus (HIV), Hepatitis (HBV, HCV).

Next, in step 126, the serology testing requirements check the donated unit against screening tests for infectious diseases that are followed based on the FDA CFR sub-part E and AABB standard and technical manual [2, 3, 56]. Step 126 in FIG. 1 shows the serology testing process consisting of screening tests and confirmatory tests. Also, FIG. 1 shows the deferral period as one of the requirements assigned to a donor if the serology testing results in a positive outcome. Identifying the donor's blood group is another sub-process shown in light green in box step 128 in FIG. 1. There are several blood group systems, where the most common one is ABO, which were used. In ABO testing, the donor's blood can be interpreted as either blood type A, B, AB, or O. This is done by detecting the presence of ABO antigens on the red cells and the absence of their corresponding antibodies in the plasma (using so-called forward and reverse blood grouping). However, in some situations, a person can develop an unexpected antibody against antigens in the other blood group systems which were modeled at a high-level in the system to capture regulatory requirement CFR 640, 660 [2, 56]. A person's immune system generates antibodies as a result of blood transfusion, pregnancy, or bone marrow/stem cell transplantation. Antibodies are generated by the human immune system to fight any foreign antigen. The blood grouping using two complex processes shown in step 128 in FIG. 1 were modeled.

The first box shows a test done using the donated samples and the second test uses a unit segment. These tests complying with FDA sub-part C and CFR640 [2] where the FDA require the tests from the donor samples. However, as an extra precaution, the ABO/Rh blood group is modeled to check the blood group in two methods shown in FIG. 1, in steps 128b and 128c. Testing for the ABO/Rh from the segment and samples ensure accurate determination of donor's blood group and minimize the possibility of a mix-up between the unit and samples during the labeling of the samples and units prior to collecting the blood from the donor. After completing the ABO/Rh processes from the segment and samples then the results are compared. Only if the ABO/Rh are the same results, then the final ABO is stored as is required and stated by the AABB standard 5.12 and 5.13 [3]. Otherwise, an investigation process is held to find any errors.

### Processing During the Post Analytical Stage

In the post-analytical phase, blood bank staff review tests performed during the analytical phase. This phase moves safe blood units to storage (FDA CFR 610.53, CFR 640, AABB standard part 5.1.8A) and unsafe blood units (units that failed tests or standards requirements) to be discarded. Units that are discarded trigger donor deferral if unsafe determination was due to positive testing for a transmissible disease. Otherwise, the unit is discarded without deferring the donor.

This process stores blood temporarily and then decomposed into different blood components such as Red Blood Cells (RBC), Platelets (PLT), Fresh Frozen Plasma (FFP) and Cryoprecipitated AHF (Cryo). Each of the processed blood components has specific storage requirements such as time, expiry date, temperature, usage and status as explained in FDA CFR 610.53, CFR 640, AABB technical manual Chapter 9 and AABB standard 5.7.4.9 [2, 3, 56]. As whole blood is directly stored when it has been processed into components (Completing the Unit Processing Step 122) it is stored in the untested storage first.

After the sample processing shown in Step 124 of FIG. 1 is completed and passed, all passing units are moved from the so-called untested storage to tested storage. These storage locations are kept in separate refrigerators. The modeled blood bank currently holds two of the following refrigerators, incubators and freezers to ensure tested and untested units are kept separated. Thirdly, any of the red box fails the unit is automatically discarded.

### Transfusion Process

The safety requirements checked during the transfusion process ensures that the recipient will receive safe blood that is compatible with the recipient's blood type, thereby decreasing the risk of transfusion related complications. Blood transfusion complications include infectious and non-infectious complications. Infectious complications include having infections (hepatitis, HIV, Syphilis, and other blood-borne infections) from blood transfusion. Non-infectious complications include hemolytic transfusion reactions, allergic transfusion reactions, febrile-non-hemolytic-transfusion reaction, TRALI, etc. In order to proceed with a safe transfusion, the transfusion process follows three phases (pre-transfusion, transfusion and post transfusion) as shown in FIG. 2.

### Processing During Pre-Transfusion

Pre-transfusion Step 106 is shown in FIG. 1. The pre-transfusion phase shown as Transfusion Request in FIG. 2 uses several tasks to check the information provided in the request form, patient history, and unit data to ensure choosing a compatible unit for the patient (AABB standard parts 5.11.1.1 and 5.28.2). The first task in TransfusionRequest process ensures that a valid request is received from a physician along with a patient blood sample. TransfusionRequest checks patient's historical data to confirm the patient's sample blood group from historical sample results if the patient had previous transfusion record (CAP TRM.30575, TRM.40300). For patients with no blood group histories, the workflow directs the user to request a second sample from the patient that is then tested to confirm the first sample for patient's blood group (AABB standard parts 5.14).

The next task is to test the patient's blood sample as stated earlier. Using specific compliance standards, the system then identifies a compatible blood unit for transfusion based on recorded patient and available units' data (electronic cross-match). The system can be designed to request also a technical (actual) cross-match in cases of unusual antibody or rare blood group incompatibility. The expiry date of the cross-matched unit is captured by the TransfusionRequest and used to ensure that the cross-matched unit cannot be held longer than three days (CAP TRM.40500, AABB standard part 5.16). The selected unit is signed-out or issued for transfusion. Urgent requests for transfusion for life saving requires skips patient blood grouping (AABB standard part 5.27).

### Processing During Transfusion

Once blood transfusion request is complete. The next step is Transfusion (step 108) in FIG. 1. The transfusion workflow design checks for specific regulatory standards that include requiring two operators (transfusionists or nurses) to verify patient ID, and matching information on the unit, dispensary report and tags (CAP TRM.40235, TRM.41300). Failure to meet this requirement stops the transfusion process if the system is also used at bedside.

Patient vital data are recorded prior to transfusion as a baseline and continuously recorded throughout the transfusion procedure to capture any transfusion reaction that may occur. The length of transfusion is also recorded and monitored to take no more than approximately 4 hours.

### Processing During Post-Transfusion

Once transfusion is complete the next step is post-transfusion (step 110). The post-transfusion phase monitors patients' vital signs and checks for any adverse reactions (CAP TRM.41475) shown in FIG. 1 in step 110. In situations where a patient experiences a transfusion reaction, the workflow allows the user to enter the symptoms to automatically interpret the type of reaction, hemolytic vs non-hemolytic, and prompts the user to collect additional relevant information or order additional tests that may help in investigating the nature of the reaction (CAP TRM.41650, TRM.42050, TRM.42150).

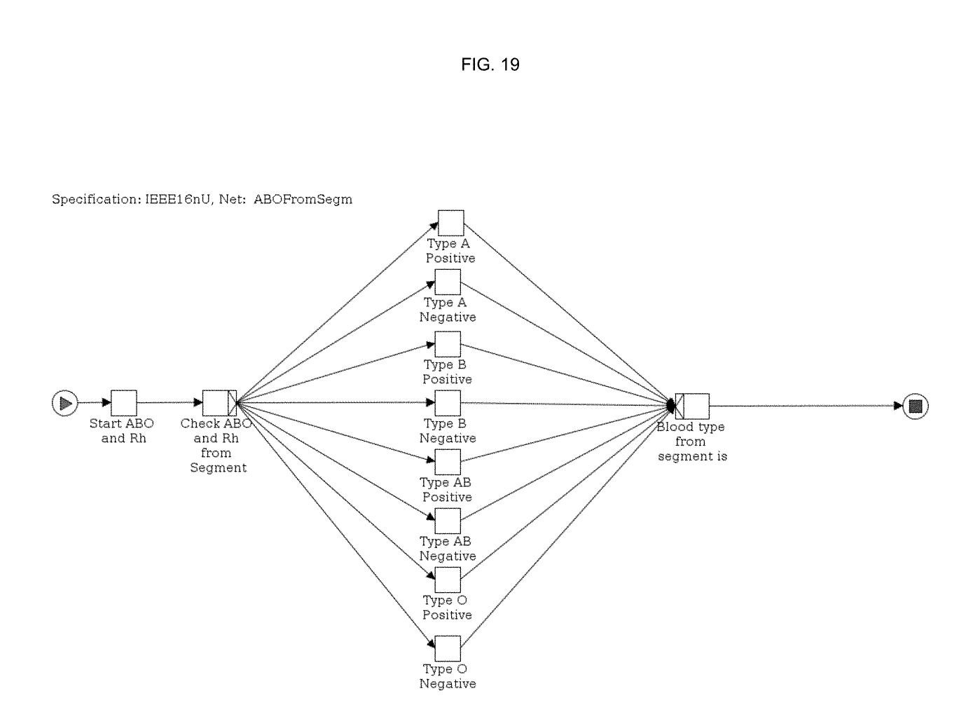

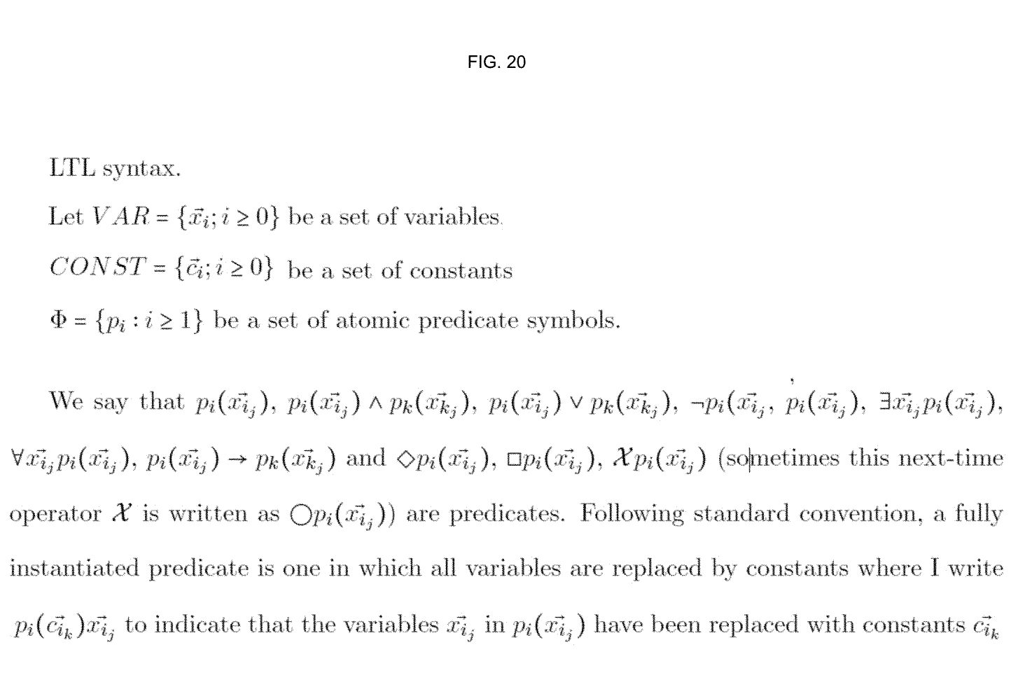

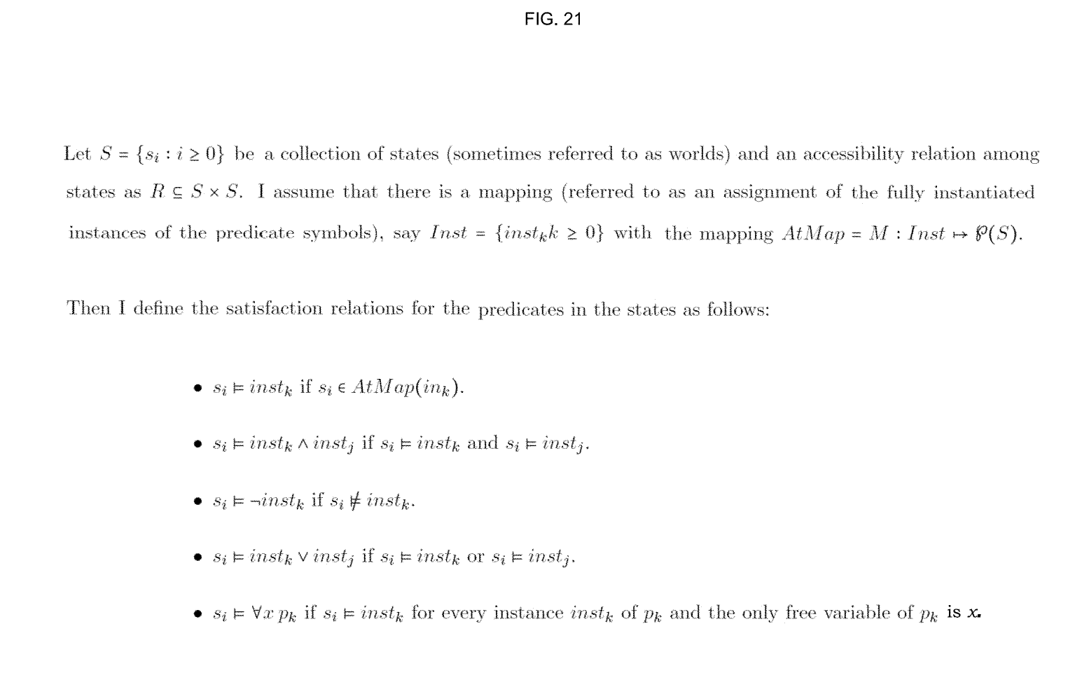

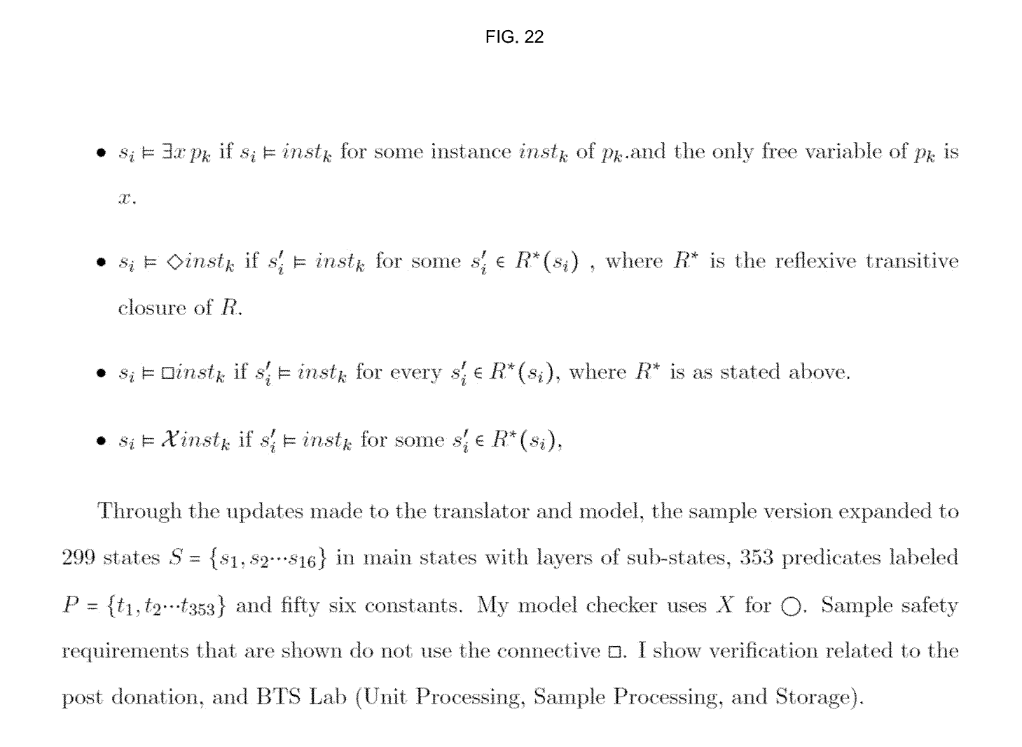

FIG. 19 illustrates a syntax (atomic predicate symbols) to be used throughout the specification and workflow diagrams. FIGS. 20 and 21 illustrate a summary of commonly used semantics of temporal logic. FIG. 22 illustrates workflows modelled as state transitions between so-called states of the blood supply chain.

Through the updates made to the translator and model, the sample version expanded to 299 states S={s1, s2 . . . s16} in main states with layers of sub-states, 353 predicates labeled P={t1, t2 . . . t353} and fifty-six constants. This model checker uses X for +. Sample safety requirements that are shown do not use the connective □. Verification related to the post donation is shown, and BTS Lab (Unit Processing, Sample Processing, and Storage).

### Verification

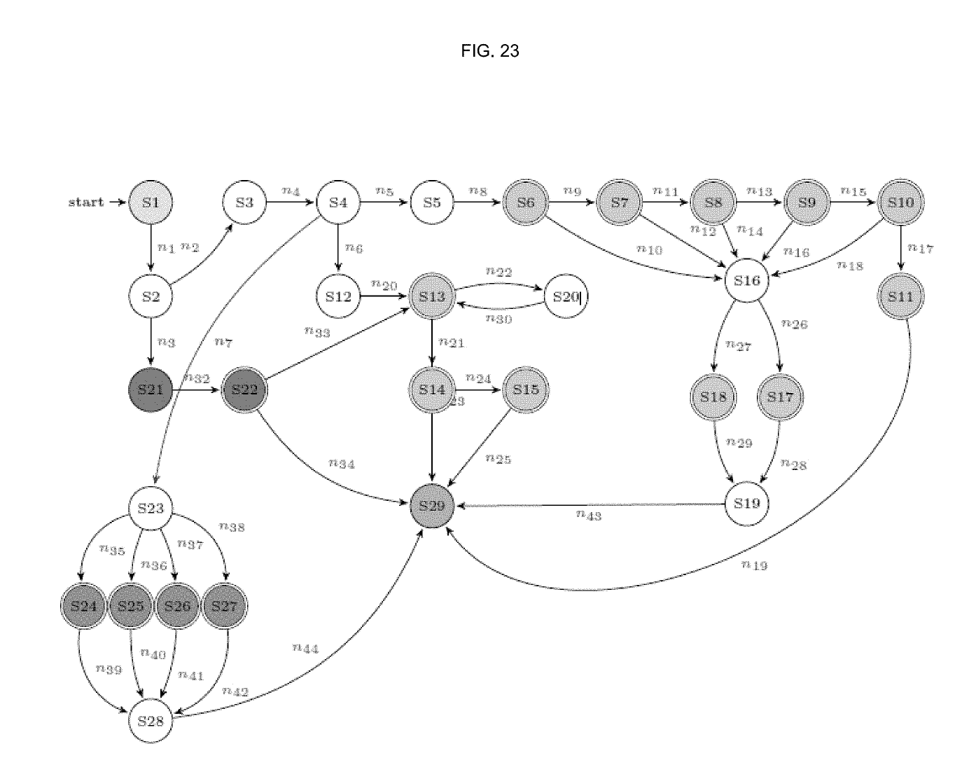

FIG. 23 illustrates workflows modelled as state transitions between so-called states of the blood supply chain.

This section describes the state space and shows that state transitions satisfy associated LTL properties that model safety regulations. Each state is described below with their transitions depending on the requirements specified in the defined safety properties. As there are 299 states, this section will only discuss states, transitions and associated LTL properties shown in FIG. 23, focusing on new states filled states (S6, S7, S8, S9, S10, S11, S13, S14, S15, S17, S18). States S21, S21 are empty states reserved to be used for future expansion to other locations. In this diagram, States such as S6, S7, S8, S9, S10, S11, S13, S14, S15, S17, S18, S22 are compound states. All states S24, S25, S26, S27 are compound states used by other states such as S9 and S12. All states ending with (1 or 0.1) are a starting state, while the colored green are ending states.

S1, S2, S3, S4, and S5: The user starts the workflow in states 51 and in S2 the user enters the country and the system will route the user country in states S3 or S21 as shown in FIG. 23. By clicking on the donation process in S4, state S4 is started followed by an automatic transition through n5 to S5 to start the donation. By clicking start transfusion process, state S12 is started followed by an automatic transition to S13. By clicking start PT testing process a transition through n7 to state S23 is started allowing the user to specify the type of test to follow which then transitions to either S24, S25, S26, S27.

S4: This state allows the user to choose their next state. The user is allowed to choose between S5, S12, and S23. Depending on the user choice the transition to the chosen state is applied.

S5: This state is an empty state. Once this state is started it automatically transitions through n8 to S6 to start the donation process.

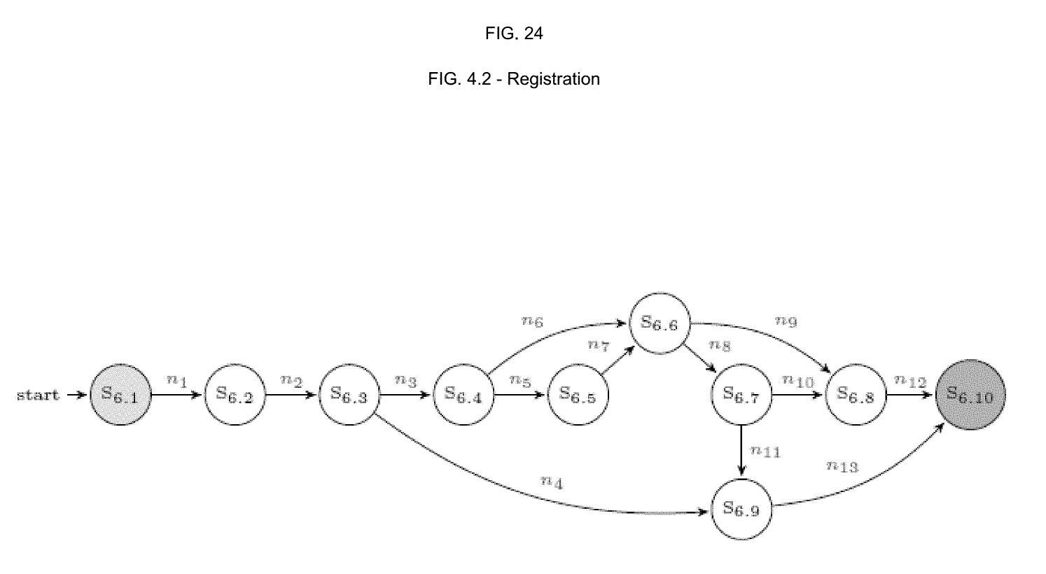

FIG. 24 illustrates registration workflow modelled as state transitions between so-called states of the blood supply chain.

S6: This is a compound state. This state checks the donor requirements for registration and outputs [donationType, donationTypeSpecific, govID, idPhoto, idExpiry, productDonated, donationInterval, donorAge]. This composite state is expanded from S6.1-56.10. The composite state then outputs the following to verify the donor registration is safe (t2, t8, t9, t10, t11, t12, t13, t14, t15, (t16, (t17, t18, (t19) or (t54, t147) on trigger transitions n9 to S13. Allowing the donor to register and donate transitioning to S7 through n9 or deferral to S16 through a transition n20.

S7: In this composite state, the system checks if the donor is suitable for the donation by checking the donor vitals. This composite state is expanded from S7.1-S7.18. The composite state then outputs the following to verify the donor is suitable and safe to donate (t2, t3, t4, t5, t6, t8, t9, t10, t19, t20, t21, t22, t23, t24, t25, t26, t27, t28, t29, t30, t31, t32, t33, t34, t35, t36, t37, t38, t39, t40, t41, t42, t45, t46, t47, t48, t49, t50, t51, t52, t53, t148) on trigger transitions n11 to S8.

S8: In this composite state, the system checks if the collection process's safety controls have been applied such as re-verifying the donor. This composite state is expanded from S8.1-58.12. The composite state then outputs the following to verify the collection is safe (t8, t9, t10, t55, t56) on trigger transitions n13 to S9. On completion, a transition to S9 through n13 for post donation. or a transition to S16 through n14 for deferral.

S9: This is a composite state shown in FIG. 23. This composite state is expanded from S9.1-S9.7. The composite state then outputs the following to verify the donor is safe after donation (t220) on trigger transitions n15 to S10. The blood bank staff enters if a donor has any adverse reactions t220 if not then a transition n15 to S10. Otherwise, transition n16 goes to state S16 for deferral. This state output [donorReactionN]

S10: This is a composite state consisting of sub-states S10.1 to S10.8 checks the blood unit for infectious diseases and donor blood grouping. The composite state then outputs the following to verify the donated unit is safe (t221, t222, t223, t224, t225, t226, t227, t228, t229, (t230, (t231, t232, (t233) or (t236, t237)))) on trigger transitions n17 to S11. This state output [HTLV, AntiHBc, HBsAG, AntiHCV, HIVNAT, HCVNAT, HBVNAT, Syphillis, AntiHIV, recordedVolume, UnitBagVolumeCap]. On completion a transition to S11 through n17 to store the unit or for deferral to S16 through n18.

S11: This is a composite state consisting of sub-states S11.1 to S11.8 stores the donated unit based on the regulatory requirements such as time, temperature, color. Also ensuring the correct labels are added if the unit has completed testing or untested. On completion a transition to S29 through n19.

S12: This is an empty state. Once it is started it automatically transition the user to state S13 through n20.

S13: This composite state checks the transfusion request, apply the transfusion and ensure the patient has no adverse reactions. Once the transfusion is complete, a transition n21 to state S14 to complete and end of transitions. This composite state consisting of sub-states S13.1 to S13.11.

S14: This composite state checks the patients to ensure all information of the unit and patient are matching. This is to make sure that the unit in hand is for the right patient. This composite state consisting of sub-states S14.1 to S14.11. On completion a transition to S15 through n24 for post transfusion.

S15: This composite state checks the patients to ensure the patient did not develop any transfusion reaction symptoms prior to releasing the patient. This composite state consisting of sub-states S15.1 to S15.7. on completion a transition to S29 through n25.

S16: In this state the system routes the deferrals based on the type of deferral as specified by the safety controls and standards. The present general inventive concept covers registration, collection, post donation and BTS lab deferrals which are shown in FIG. 23 as transitions n10, n12, n14, n16, and n18. The composite states consist of S17.1 and S17.2 for deferrals and discard units in S18.1 and S18.2

S17: This is a composite state for deferrals. This composite state consists of sub-states that show deferrals for registration, suitability, collection. On completion a transition S19 through n28.

S18: This is a composite state for discarding units. This composite state consists of an empty sub-state. As this work is relying on third party for discarding units. On completion a transition to S19 through n29.

S21: Empty state to transition to Country 2 workflow.

S22: This is a composite state Country 2 registration.

S23: This state requires the user to specify the type of proficiency testing service needed to be performed such as serology, unit crossmatch, check donor blood group and check patient blood group. Based on the user input a transition occurs to one or more of the following states S24, S25, S26, S27.

S24: This is a composite state that performs serology testing from S10.4.5.1-S10.4.5.10. On completion a transition to S28 through n39.

S25: This is a composite state that performs unit crossmatch and transitions from S13.9.1-S13.9.7. On completion a transition to S28 through n40.

S26: This is a composite state that performs donor blood grouping and transitions from S10.4.4.1-S10.4.4.8. On completion a transition to S28 through n41.

S27: This is a composite state that performs patient blood grouping and transitions from S13.14.1-S13.14.13. On completion a transition to S28 through n42.

S28: This is an empty state that automatically transitions to S29. On completion a transition to S29 through n44.

S29: This is the final state for all transitions. This is an output condition showing completion of all states and no transitions left.

In FIG. 24, state S6 decomposes into 10 sub-states labeled S6.1 through 56.10. This complex state routes the donor correctly through checking any donations history. Also, ensuring all donors (new or returning) are safe to donate through adding an extra checkpoint to check for donation intervals for specific donors.

S6.1 This start state of the sub-workflow, where the completion of S5 trigger S6 to start only if the donation starts. This transition takes the system from state S6 to state S6.1.

S6.2 This is an empty state. Once it is started it transitions to S6.3.

S6.3 This state requires the user to verify the donor through a Boolean of [govID, idPhoto, idExpiry]. Only if the donor passes the verification a transition to S6.4. If not, then the user is transitioned to S6.9. The state then outputs the following to verify the donor identity (t126, t27, t28, t138) on trigger transitions n3 to S6.4.

S6.4 This state requires the user to input boolean [newDonor]. On trigger of (t153) a transition n5 to S6.5. If the donor is new a transition to S6.5 to register the donor. Otherwise a transition to S6.6 to check-in the donor.

S6.5 This state the user enters [DonorID, donorName]. Once completed a transition to S6.6 to check-in the donor.

S6.6 This state is to check-in the Donor, the user enters [donationtype, donationtypespecific, previouslydonated]. if previously donated then transition to S6.7 otherwise S6.8.

S6.7 Check donation interval, the user enters [productDonated, donationinterval]. If the donor donated PLT “2 or PLA “28 or RBC “56 or DRBC “112 then transition to S6.8. On trigger of (t131, t132, t133, t135, t136, t37) a transition n10 to S6.8. otherwise, transition to S6.9 to defer the donor.

S6.8 This state outputs to the user all the data entered to the user to provide education material to the donor. Then a transition to S6.10.

S6.9 This state output to the user all the data enter to donors who have been deferred due to invalid ID or not completing the donation interval. Completion of this state transition to S6.10.

56.10 This is the end of the sub-state, it outputs [donationType, donationTypeSpecific, govID, idphoto, idExpiry, productDonated, donationinterval, donorAge] to the main state S6.

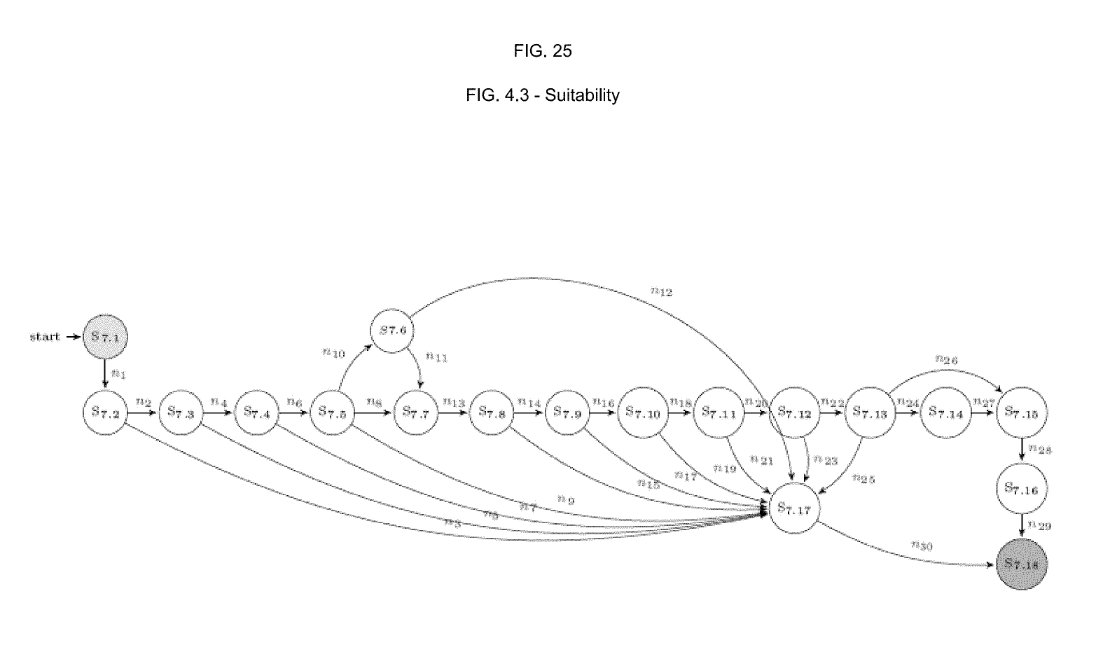

FIG. 25 illustrates suitability workflow modelled as state transitions between so-called states of the blood supply chain;

The decomposition of composite state State 7 is shown in FIG. 25 where the safety property associated with the first row of Table C.2 holds in state S7.1. Because S7.1 is the beginning state of sub-workflow where the system imports the donation type and donation specific [donationTypeN, donationTypeSpecific] starts in state S7. The decomposition takes the system through 18 subsequent states numbered S7.1 through S7.18. It is modeled as transition n9 that is triggered by action start and goes from state S6 to S7 in FIG. 23. The decomposition of composite state in State 7 in FIG. 23 is illustrated in pink color with the third property holding at S7.1 explained in Table C.2

S7.1: The start state of the sub-workflow, where the completion of S6 trigger S7 to starts only if the donor passes the registration based on the conditions explained above. This transition, takes the system from state S7 to state S7.1.

S7.2: This state requires the user to re-verify the donor through a Boolean of [govID, idPhoto, idExpiry]. Only if the donor passes the verification a transition to S7.3 n2.

If not, then the user is transitioned back to states S17 through n3 as the donor is deferred. On trigger of (t109, t110, t111) a transition to S7.3 on n2.

S7.3 In this state is entered if the system received blood donation consent from the donor [consent], modeled as the transition n4 that takes the system from state S7.3 to state S7.4 triggered by the action t112. On trigger of t112 a transition to S7.4 on n4.

S7.4: The user enters [medication and lastDosageDays] to proceed. It is modeled as transition n6 taking the system from state S7.4 to state S7.5 triggered by actions t84 . . . t98

S7.5: The system imports the donation type and its details that are specified in state

S6.6 [donationType and donationTypeSpecific], checks if the donation type specific is a directed or autologous donation. This is modeled as transition n8 that takes the system from state S7.5 to state S7.7 automatically. If not, other types of donations will transition triggered by action n10 to S7.6.

S7.6 The user will check and enter if the donor received [physicianRequest] to the system. If the condition in t114 is satisfied transition n11 takes the system to state S7.7. If not, transition n12 takes the system to state S17.

S7.7: The user or blood technician will check the donor's temperature and enter it in [temp]. The system then automatically transitions the user to state S7.7 triggered by the action t113. If not, then the system will automatically transition to state S17 due to action n13.

S7.8 The system will import [donationTypeSpecific] from the S6.6 using transition n14. The user is also required to enter the hemoglobin level [hgb]. The system then automatically transitions the user to state S7.9 triggered by t81 . . . t83, t123 and t124. If not, then the system state transitions to S17 due to transition n15.

S7.9 The user will check and enter the donor [systolicPressure] to the system. Only if the condition in t115 is satisfied, a transition of n17 to the next state S7.10 occurs. If not, then the transition n17 will change the system state to S17.

S7.10 The user will check and enter the donor [diastolicPressure]. If the condition in t116 is satisfied, the transition n18 will take the system to state S7.11. If not, then the transition n19 takes the system to state S7.17.

S7.11 The user will check and enter the donor [armSkinDiseaseFree] to the system. If the condition in t117 is satisfied, a transition of n20 to the next state S7.12 occurs. Else, a transition of n21 to 57.17 occurs.

S7.12 The user will check and enter the donor [pulse] to the system. If the condition in between t118 and t119, a transition of n22 to the next state S7.13 occurs. Else, a transition of n23 to 57.17 occurs.

S7.13 The user will check and enter the donor [weight, sex, donationType] to the system. If the conditions in t120 . . . t122, t107 t108 are satisfied, a transition n24 takes the system to state S7.14 or 57.15. If the donor is donating platelets then a transition to S7.14, else a transition S7.15 for other donation types. If the state does not satisfy the condition, the transition n25 takes the system state to S7.17.

S7.14 The user will check and enter the donor [PLTcount] to the system. If the condition in t?? is satisfied the transition n27 takes the system to state S7.15.

S7.15 The user will enter boolean answers for [Q1-Q49] the questionnaire. Completing this state result in a transition to S7.16.

S7.16 This is an empty state, to show the end of the suitability for the donor.

S7.17 This state inputs all data entered by the deferred user. On completion of this state, a transition to S7.18

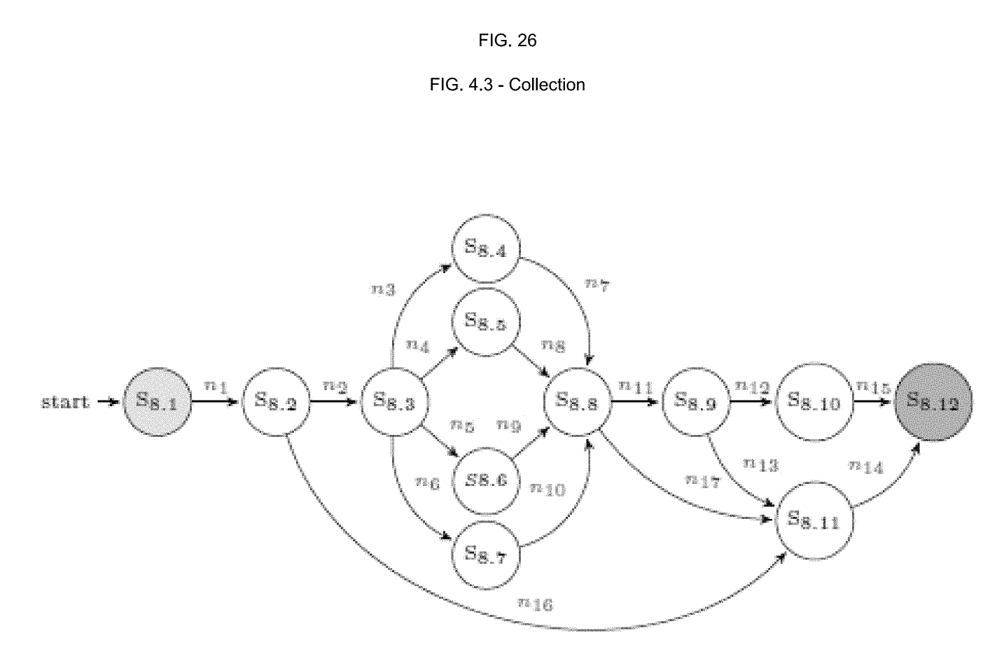

FIG. 26 illustrates collection workflow modelled as state transitions between so-called states of the blood supply chain.

S7.18 This is the final state of this composite state. On completion of this state a transition from S7.18 to S7. The composite state in State 8 in FIG. 23 in pink color with the fourth property holding at S8.1 explained in Table C.2. FIG. 26, this is the decomposition of State 8:

S8.1 The start state of the sub-workflow, where the completion of S7 trigger S8 to starts only if the donor passes the suitability based on the conditions explained above. This transition takes the system from state S8 to state S8.1.

S8.2 The user is required to re-verify the donor's identity using attributes modeled as Booleans [govID, idPhoto, idExpiry]. Only if the donor passes the verification, FIG. 26 on trigger of t142, t143, t144 a transition to S8.3 n2 occurs. If not, then the user is transitioned to S11 through n16 occurs.

S8.3 The system will route the user to the correct blood unit set depending on the type of donation through [donationType]. On trigger of t139, t140, t141 a transition to states S8.4 through n3, S8.5 through n4, S8.6 through n5, S8.7 through n6

S8.4 The user is required to enter [lotNo, serialNo, checkLabel, anticoagulant, anticoagulantVolume, UnitbagVolumeCAP] for whole blood. On completion, the user is transitioned to S8.8 through n7.

S8.5 The user is required to enter [lotNo, serialNo, checkLabel, anticoagulant, anticoagulantVolume, UnitbagVolumeCAP] for plasmapheresis. On completion, the user is transitioned to S8.8 through n8.

S8.6 The user is required to enter [lotNo, serialNo, checkLabel, anticoagulant, anticoagulantVolume, UnitbagVolumeCAP] for plateletpheresis. On completion, the user is transitioned to S8.8 through n9.

S8.7 The user is required to enter [lotNo, serialNo, checkLabel, anticoagulant, anticoagulantVolume, UnitbagVolumeCAP] for RBC pheresis. On completion, the user is transitioned to S8.8 through n10.

S8.8 The user has to enter attributes [FindVein, armScrub]. On trigger of t145 and t146 the system state transitions to S8.9 through n11. Otherwise, the system state transitions to states S8.11 through n17 in order to defer the donor.

S8.9 The user monitors the donor and enter the following [donorReaction, donorReactionDescr, collectionStartTime, collectionDate]. As a consequence, the system transition n12 to state S8.10 on trigger of t145, t146.

S8.10 The user monitors the donor and enter the following [donorReaction, donorReactionDescr, collectionEndtTime]. As a consequence, the system transition n15 to state S8.12.

S8.11 This state output to the user all the data entered previously for this donation. On completion, a transition to S8.12.

S8.12 This is the final state of this sub-state. A transition the higher state S8 and an output of all the user input of [govID, idPhoto, idExpiry, findVein, armScrub, lotNO, serialNo, checkLabel, donorReaction, donorReactionDescr, unitBagVolumeCap, anticoagulant]

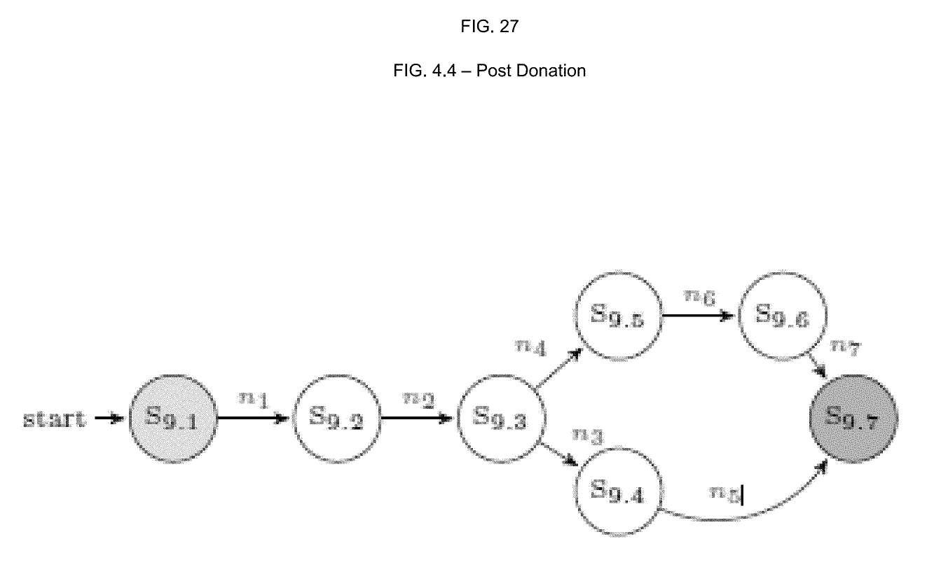

FIG. 27 illustrates post donation workflow modelled as state transitions between so-called states of the blood supply chain.

In FIG. 27, state S9 decomposes sub-states S9.1 through S9.7 where donors are monitored to ensure that no adverse reactions occur and if they do all symptoms should be recorded.

S9.1 The start state of the sub-workflow, where the completion of S8 trigger S9 to starts only if the donor passes the collection based on the conditions explained above.

This transition takes the system from state S9 to state S9.1.

S9.2 This is an empty state. On completion, it automatically transitions to S9.3.

S9.3 This state the user is required to enter a boolean into the system [adverseReaction] if the donor is experiencing any adverse reaction. A transition to S9.4 on n3 for donors experiencing an adverse reaction.

S9.4 This state, the user enters [typePD, delayedPD, dTimePD, reactionCategoryPD]. On completion a transition to S9.7

S9.5 This is an empty state. On trigger a transition to S9.6

S9.6 This is an empty state. On trigger, a transition to S9.7 as the user is required to provide the donor instruction sheet for post phlebotomy and adverse events.

S9.7 This is the final state of this sub-state. A transition the higher state S9 and an output of all the user input of [adverseReaction, typePD, delayedPD, reactionCategoryPD]

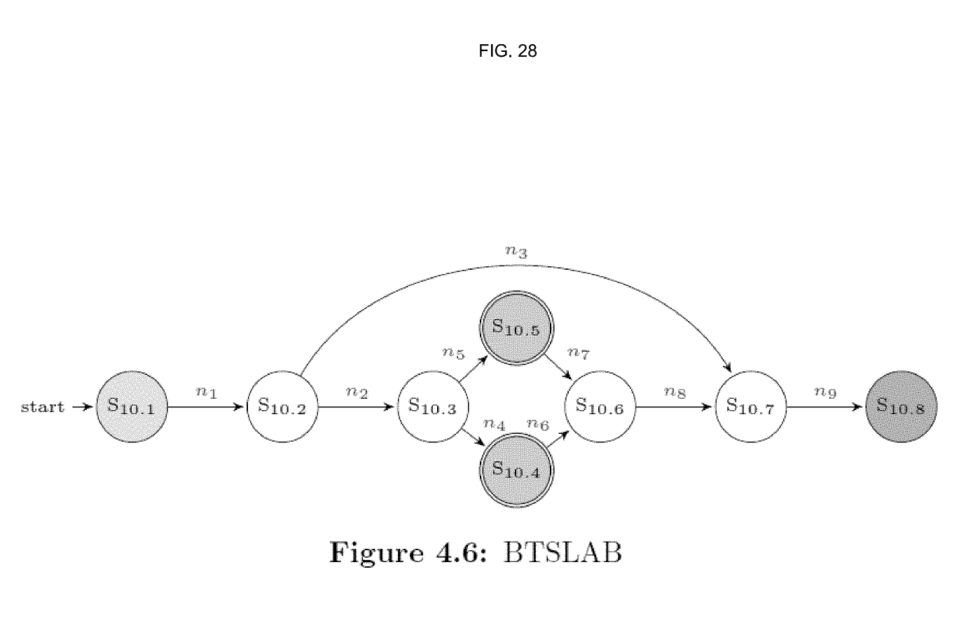

FIG. 28 illustrates BTSLAB workflow modelled as state transitions between so-called states of the blood supply chain.

In FIG. 28, the high level state BTSLAB S10 decomposes into sub-states S10.1 through S10.10. The composite state S10 checks the donated unit and process the unit into different products. This state consists of two sub-states S10.4 and S10.5. These states show the transitions needed for testing blood for infectious diseases/blood grouping and processing the unit to different blood products.

S10.1 This state starts the sub-states. When entered, it imports [donationType, donationTypeSpec, UnitBagVolumeCap, Anticoagulant, TransRequest, PatientBlood-Group, finalABOTR] to be utilized in the sub-states. Then it transitions to state S10.2.

S10.2 In this state, the blood unit is received and inspected, the user enters [leaking, matchRegisF, UnitArrivalTime, BloodColor, DonationID, Temp, bForProcessingTo, UnitWeight]. Other information are imported from the main state such as [donation-Type, donationTypeSpec, HistoryABO, routineTrans]. If the entered data meets the condition based on the type of donation and unit (temperature, weight, processingto, etc.) a transition to S10.3 on n2 on trigger of t154, t155, t156, t157, t158, t159, t160, t161, t162, t163. Else a transition to S10.7 on n3.

S10.3 This is an empty state as the user is not required to enter or interact with the system. As S10.2 completes, this state start transitioning in to states S10.4 and S10.5 simultaneously.

S10.4 This is a composite state for sample processing. It input [DonationID] and after processing checks the samples for infectious diseases and finding donor blood group then outputs [RIBA, HBVNAT, finalABOType, antiHIV, HCVNAT, HIVNAT, HBsAG, antiHc, HTLV, Syphillis, Treponomal, antiHCV]. On trigger (t165 through t175) transition n6 to state S10.6.

S10.5 This is a composite state for unit processing. It imports [bForProcessingTo, DonationID, anticoagulant, unitBagVolumeCap] from this net to S10.5 composite states. Depending on the donation, it process the unit in the substates then the results outputs in this state [ProductID01-03, ProductRecordedVolume01-03, productID01-03]. On trigger (t159, t154, t177, t181) a transition n7 to S10.6.

S10.6 This is a composite state that checks if the unit of blood is safe after the unit has been processed and checked for serology by checking the following requirement [unitTested] if they meet the safety standards and regulations a transition n8 to state S10.7. Not passing any of the safety requirements transitions n9 to state S10.8 where it collects the summary of this sub-state.

S10.7 an empty state that transitions to state S10.8 for storage needed for transfusion.

S10.8 This is the final state of this sub-state. A transition the higher state S10 and an output of all the user input of [Leaking, Unitweight, successionID, MatchRegis, Temp, UnitArrivalTime, ForProcessingTo, bloodColor, HTLV, AntiHBc, HBsAG, AntiHCV, HIVNAT, HCVNAT, HBVNAT, Syphillis, AntiHIV, RIBA, Treponomal, recordVolume, FinalABOType, ProductID01-ProductID03, unitTested]

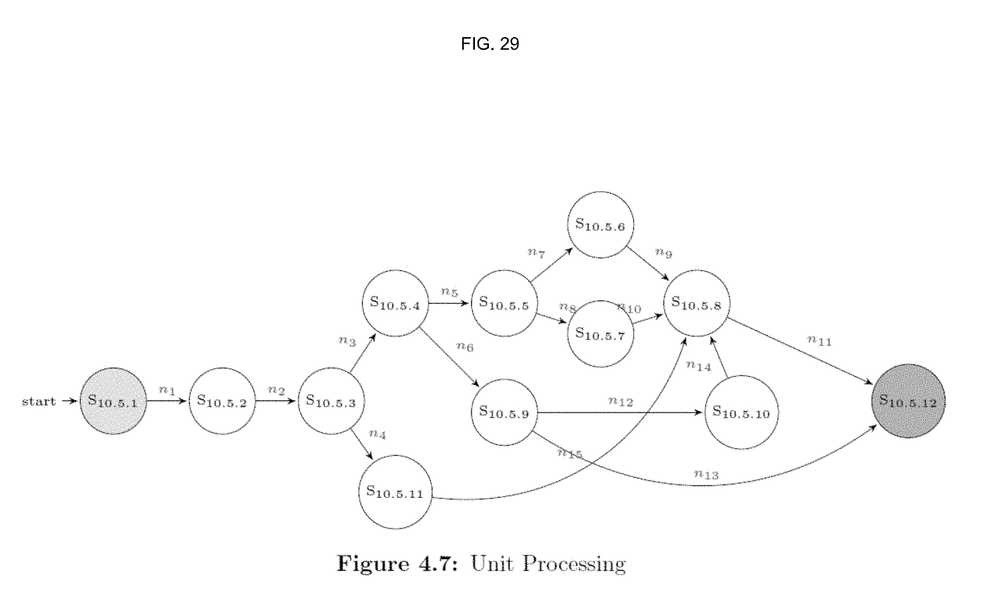

FIG. 29 illustrates unit processing workflow modelled as state transitions between so-called states of the blood supply chain.

FIG. 28 illustrates the composite state S10.5. This state checks the donated unit upon receival at the laboratory. This section describes the donor unit processing. FIG. 29 shows the decomposition of S10.5: is a composite state consists of states S10.5.1 through S10.5.12 used to model unit processing.

S10.5.1: This state start gets the inputs [donatioType, donationTypeSpecf, donationID, UnitBagVolumeCap, ForProcessingTo] from state S10.5.

S10.5.2: This is an empty state that transitions to S10.5.3.

S10.5.3: This state input [donationType] to check if the donation is for Whole blood or Apheresis. Whole blood donations are transitioned to state S10.5.4 on n3 on trigger of t184. while Apheresis are transitioned to state S10.5.11 on n4.

S10.5.4: This state checks if the donated unit of blood is within the regulated FDA safety standard for the donated unit volume, depending on the unit bag capacity used which is transitioned to state S10.5.5 on n5 on trigger of t185, t186, t187, t188, t189, t190, t191. Donated units with higher or lower than standard volume are transitioned to state S10.5.9 on n6.

S10.5.5: This state checks the blood components the user specified for the whole blood processing. The user is required to input [ForProcessingTo] the system checks this variable to identify if blood to be processed as RBC FFP PLT transitions to state S10.5.6 or RBC and Cryo that transitions to state S10.5.7 on n8. On trigger (t191) transition n7 to state S10.5.6.

S10.5.6: This state creates new products for RBC, FFP, PLT and checks the volume of each bag and then outputs [productID01-03, processedTo01-03, productRecordedVolume01-03].

S10.5.7: This state creates new products for RBC and Cryo and checks the volume of each bag then it output the following [productID01-02, processedTo01-02, productRecordedVolume01-02]. S10.5.8: This is an empty state that transitions to S10.5.12

S10.5.9: This state checks the donated unit volume. On input [unitBagVolumeCap, recordedVolume], it compares if the unit capacity versus the recorded volume if it is lower it transition to state S10.5.10 to created packed RBC. Higher volume transition to state S10.5.12, an empty state that completes the unit processing and transitions to state S10.5.8. On trigger (t186, t187, t189, t190, t193, t194, t196, t197) transition n108 to state S10.5.10.

S10.5.10: This state creates packed RBC and records the volume and productID and outputs [productRecordedVolume01, productID01]

S10.5.11: This state registers apheresis products. Currently, an empty state left for future expansion.

S10.5.12: This end of the sub-process outputs [finalABO, donationID, unitTested, processedTo01-03, productID01-03, anticoagulant, productVolume01-03] to state S10.5

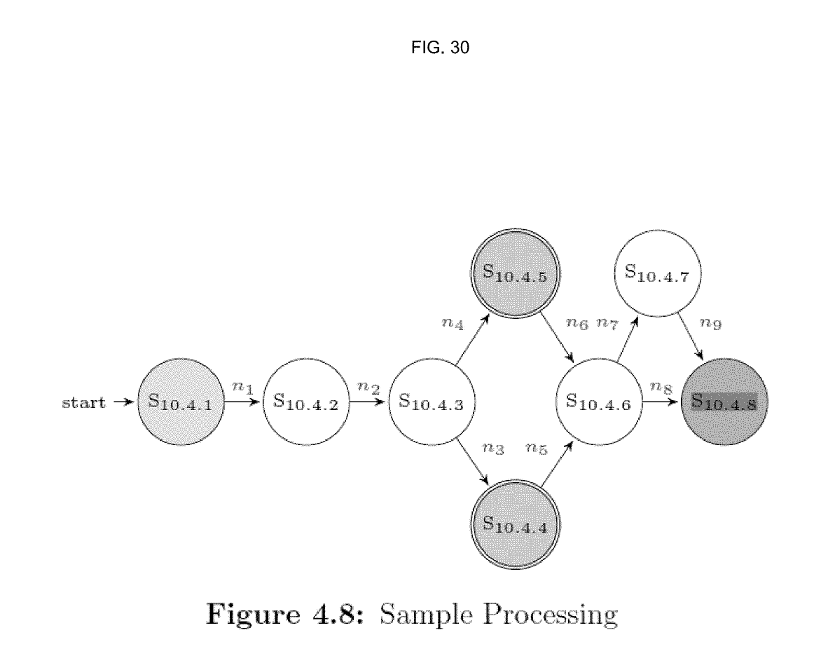

FIG. 30 illustrates sample processing workflow modelled as state transitions between so-called states of the blood supply chain.

FIG. 28 shows the composite state S10.4. This state checks the donation samples for infectious diseases and donor blood group. This section describes the donor sample testing. FIG. 30 shows the decomposition of state S10.4: is a composite state consists of states S10.4.1 through S10.4.8 used to model sample Processing.

S10.4.1: This state starts the sub-states. When S10.4 is entered this state it imports [donationID] to be utilized in the sub-states. Then it transitions to S10.4.2.

S10.4.2: This is an empty state that transitions into S10.4.3. two composite states S10.4.3 and S10.4.4 simultaneously.

S10.4.3: This is an empty state. On start it triggers the two composite states S10.4.3 and S10.4.4 simultaneously.

S10.4.4: This composite state checks the donor's blood group the presence of ABO antigens on the red cell and the absence of their complimentary antibodies in plasma cells. This state outputs [finalABOType].

S10.4.5: This composite state checks for infectious diseases serology from the donors sample. This state inputs [donationID] and output [RIBA, HBVNAT, antiHIV, HCVNAT, HIVNAT, HBsAG, antiHc, HTLV, Syphillis, Treponomal, antiHCV]. On trigger (t218-t227) transition n6 to state S10.4.6

S10.4.6: This state checks the outputs from states S10.4.4 and S10.4.5 by checking infectious diseases in all of the serology tests are negative which then transitions to state S10.4.8 on n8. A positive serology will transition to state S10.4.7 on n7. These transitions are triggered by t198, t199, t200, t201, t202, t203, t204, t205, t206, t207, t208.

S10.4.7: This is an empty state that on start input n123 transitions to S10.4.8.

S10.4.8: This state end a composite state and output [finalABOType, RIBA, HBVNAT, antiHIV, HCVNAT, HIVNAT, HBsAG, antiHc, HTLV, Syphillis, Treponomal, antiHCV] to state S10.4.

FIG. 30 shows the composite state S10.4.5. This state checks the donation samples for infectious diseases. This section describes the donor sample testing.

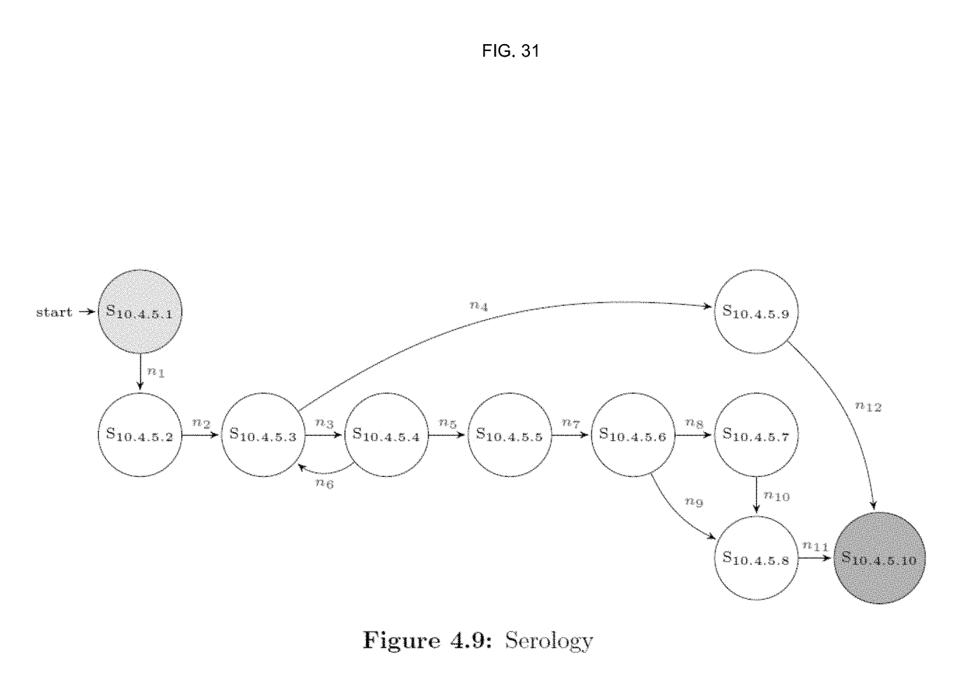

FIG. 31 illustrates serology workflow modelled as state transitions between so-called states of the blood supply chain.

FIG. 31 shows the decomposition of state S10.4.5: is a composite state consists of states S10.4.5.1 through S10.4.5.10 used to model serology infectious disease testing.

S10.4.5.1: This is an empty state that decomposes when S10.4.5 is started. This state input [donationIDSero]. On completion a transition to S10.4.5.2.

S10.4.5.2: This is an empty state. On start a transition to S10.4.5.3.

S10.4.5.3: This state the user test the donated blood and enter a boolean if positive for each of [HTLV, AntiHBc, HBsAG, AntiHCV, Syphillis, AntiHIV]. On completion a transition to 510.4.5.4 on n3 if one or more tests are positive or S10.4.5.9 on n4 if all tests are negative. These transitions are triggered by t257, t258, t259, t260, t265, t264.

S10.4.5.4: This state checks if this is the first screening test or second by entering a boolean [secondScreeningTest]. On completion a transition to 510.4.5.5 on n7 to check previous donations tests or S10.4.5.3 on n6 if false to redo the screening test.

S10.4.5.5: This is an empty state saved for future implementation to connect with the database for donation history. On start an automatic transition to S10.4.5.6 on n7.

S10.4.5.6: This is a composite state to dispose the unit through a third party. On completion a transition to S10.4.5.8 on n9 if donor blood showed positive for HTLV or AntiHBc. Else S10.4.5.7 on n8 to do confirmatory testing.

S10.4.5.7: This state test the donated blood through a different methodology to confirm test results. After the user completes the confirmatory testing, the user enters [HIVNAT, HCVNAT, HBVNAT, RIBA, Treponomal]. On completion a transition to S10.4.5.8 on n10.

S10.4.5.8: This state inputs all of the previously entered results for deferrals [donation-IDSero, HTLV, AntiHBc, HBsAG, AntiHCV, Syphillis, AntiHIV, HIVNAT, HCVNAT, HBVNAT, RIBA, Treponomal]. On completion transition to S10.4.5.10 on n11.

S10.4.5.9: This state inputs all of the previously entered results for passing all tests [donationIDSero, HTLV, AntiHBc, HBsAG, AntiHCV, Syphillis, AntiHIV, HIVNAT, HCVNAT, HBVNAT, RIBA, Treponomal]. On completion transition to S10.4.5.10 on n12.

S10.4.5.10: This is the end state it outputs [HTLV, AntiHBc, HBsAG, AntiHCV, Syphillis, AntiHIV, HIVNAT, HCVNAT, HBVNAT, RIBA, Treponomal] and transitions to S10.4.5.

FIG. 30 shows the composite state S10.4.4. This state checks the donated sample at the laboratory for blood grouping test. This section describes the donor blood grouping process.

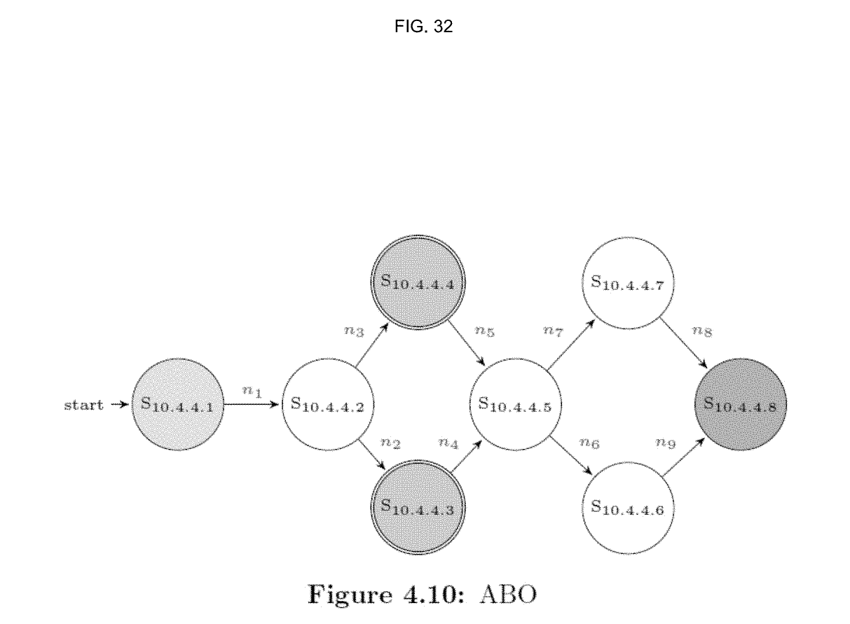

FIG. 32 illustrates ABO workflow modelled as state transitions between so-called states of the blood supply chain.

FIG. 32 shows the decomposition of state S10.4.4: is a composite state consists of states S10.4.4.1 through S10.4.4.8 used to model ABO testing.

S10.4.4.1: This state is started by a trigger from 10.4.4. This trigger starts this state which transitions to S10.4.4.2.

S10.4.4.2: This state is an empty state. On trigger it automatically transitions to S10.4.4.3 and S10.4.4.4 simultaneously to check ABO and Rh using two methods.

S10.4.4.3: This state is a composite state. on start it decomposes to S10.4.4.3.1 to S10.4.4.3.13 to check the ABO from the sample.

S10.4.4.4: This state is a composite state. on start it decomposes to S10.4.4.3.1 to S10.4.4.3.13. To check the ABO and Rh from Segment.

S10.4.4.5: This state input the following from the composite states S10.4.4.3 and S10.4.4.4 for [segBloodType, BloodTypeSample, RhSeg, RhSam]. Check if the ABO tests match if ABO from S10.4.4.3 is equal to ABO from S10.4.4.4 match. A match in the ABO triggers a transition to S10.4.4.6 on n6. Else, a transition to S10.4.4.7 on n7 for further investigation.

S10.4.4.6: This state input [bloodFinalType, RhFinal]. A transition to 510.4.4.8.

S10.4.4.7: This is an empty state, can be use for future work that shows the investigation transitions.

S10.4.4.8: This is the end state of this sub-state. A transition to S10.4.4, with the output [bloodFinalType, RhFinal]

FIG. 32 shows the composite state S10.4.4.3 in pink. This state checks the donated sample at the laboratory for blood grouping test. This section describes the donor blood grouping process from sample.

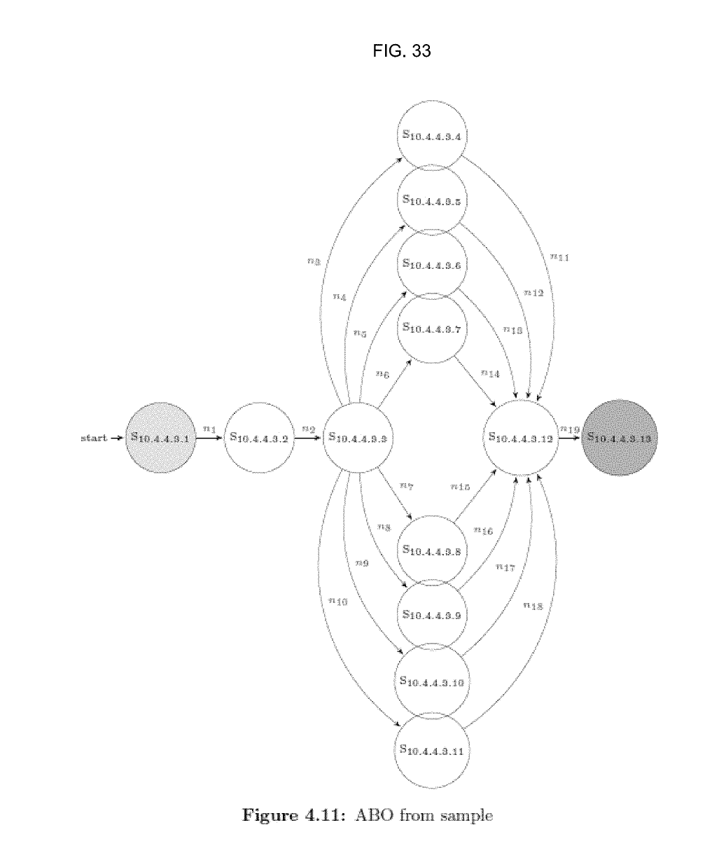

FIG. 33 illustrates ABO from sample workflow modelled as state transitions between so-called states of the blood supply chain.

FIG. 33 shows the decomposition of state S10.4.4.3: is a composite state consists of states S10.4.4.3.1 through S10.4.4.3.13 used to model ABO testing from the sample.

S10.4.4.3.1: This is an empty state. It automatically transitions on start to S10.4.4.3.2.

S10.4.4.3.2: This is an empty states that start ABO from sample and transitions to S10.4.4.3.3 on start.

S10.4.4.3.3: This state, the user enters [samAntiA, samAntiB, sam1anti1AB, samD6n, samCTL, samA1, samBCell] into the system. On trigger of t238 to t256 a transition to S10.4.4.3.4 or S10.4.4.3.5 or S10.4.4.3.6 or S10.4.4.3.7 or S10.4.4.3.8 or S10.4.4.3.9 or S10.4.4.3.10 or S10.4.4.3.11.

S10.4.4.3.4: On start the system input [samBloodABO, rhLocal] and output [samBlood-ABO, rhLocal]. To interpret the type of blood Type A POS. A transition to S10.4.4.3.12.

S10.4.4.3.5: On start the system input [samBloodABO, rhLocal] and output [samBlood-ABO, rhLocal]. To interpret the type of blood Type A NEG. A transition to S10.4.4.3.12.

S10.4.4.3.6: On start the system input [samBloodABO, rhLocal] and output [samBlood-ABO, rhLocal]. To interpret the type of blood Type B POS. A transition to S10.4.4.3.12.

S10.4.4.3.7: On start the system input [samBloodABO, rhLocal] and output [samBlood-ABO, rhLocal]. To interpret the type of blood Type B NEG. A transition to S10.4.4.3.12

S10.4.4.3.8: On start the system input [samBloodABO, rhLocal] and output [sam-BloodABO, rhLocal]. To interpret the type of blood Type AB POS. A transition to S10.4.4.3.12

510.4.4.3.9: On start the system input [samBloodABO, rhLocal] and output [sam-BloodABO, rhLocal]. To interpret the type of blood Type AB NEG. A transition to S10.4.4.3.12

S10.4.4.3.10: On start the system input [samBloodABO, rhLocal] and output [sam-BloodABO, rhLocal]. To interpret the type of blood Type 0 POS. A transition to S10.4.4.3.12

S10.4.4.3.11: On start the system input [samBloodABO, rhLocal] and output [sam-BloodABO, rhLocal]. To interpret the type of blood Type 0 NEG. A transition to S10.4.4.3.12

S10.4.4.3.12: This state input [samBloodType, FinalRh] and output [samBloodType, FinalRh]

S10.4.4.3.13: This is the end of the transitions of this sub-state and it output [sam-BloodType, FinalRh] the final ABO, Rh to S10.4.4.3

FIG. 32 shows the composite state S10.4.4.4 in pink. This state checks the donated unit segment at the laboratory for blood grouping test. This section describes the donor blood grouping process from the unit segment.

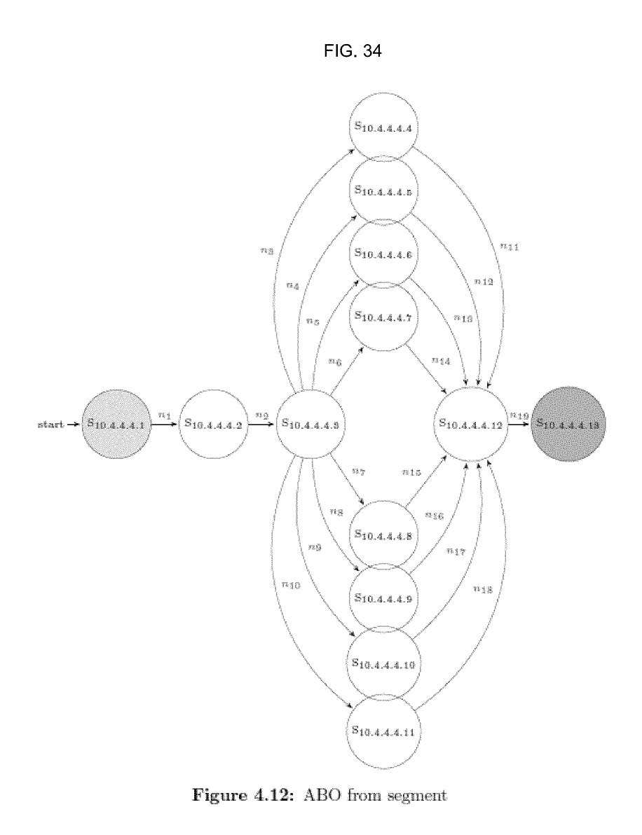

FIG. 34 illustrates ABO from segment workflow modelled as state transitions between so-called states of the blood supply chain.

FIG. 34 shows the decomposition of state S10.4.4.4. S10.4.4.4 is a composite state consists of states S10.4.4.4.1 through S10.4.4.4.13 used to model ABO testing from segment.

S10.4.4.4.1: This is an empty state. It automatically transitions on start to S10.4.4.4.2.

S10.4.4.4.2: This is an empty states that start ABO from segment and transitions to S10.4.4.4.3 on start.

S10.4.4.4.3: This state, the user enters [segAntiA, segAntiB, seg1anti1AB, segD6n, segCTL] into the system. On trigger of t279 to t308 a transition to S10.4.4.4.4 on n3 or S10.4.4.4.5 on n4 or S10.4.4.4.6 on n5 or S10.4.4.4.7 on n6 or S10.4.4.4.8 on n7 or S10.4.4.4.9 on n8 or S10.4.4.4.10 on n9 or S10.4.4.4.11 on n10.

S10.4.4.4.4: On start the system input [bloodGroupLocal, rhLocal] and output [blood-GroupLocal, rhLocal]. To interpret the type of blood Type A POS. A transition to S10.4.4.4.12 on n11.

S10.4.4.4.5: On start the system input [bloodGroupLocal, rhLocal] and output [blood-GroupLocal, rhLocal]. To interpret the type of blood Type A NEG. A transition to S10.4.4.4.12 on n12.

S10.4.4.4.6: On start the system input [bloodGroupLocal, rhLocal] and output [blood-GroupLocal, rhLocal]. To interpret the type of blood Type B POS. A transition to S10.4.4.4.12 on n13.

S10.4.4.4.7: On start the system input [bloodGroupLocal, rhLocal] and output [blood-GroupLocal, rhLocal]. To interpret the type of blood Type B NEG. A transition to S10.4.4.4.12 on n14.

S10.4.4.4.8: On start the system input [bloodGroupLocal, rhLocal] and output [blood-GroupLocal, rhLocal]. To interpret the type of blood Type AB POS. A transition to S10.4.4.4.12 on n15.

S10.4.4.4.9: On start the system input [bloodGroupLocal, rhLocal] and output [blood-GroupLocal, rhLocal]. To interpret the type of blood Type AB NEG. A transition to S10.4.4.4.12 on n16.

S10.4.4.4.10: On start the system input [bloodGroupLocal, rhLocal] and output [blood-GroupLocal, rhLocal]. To interpret the type of blood Type 0 POS. A transition to S10.4.4.4.12 on n17.

S10.4.4.4.11: On start the system input [bloodGroupLocal, rhLocal] and output [blood-GroupLocal, rhLocal]. To interpret the type of blood Type 0 NEG. A transition to S10.4.4.4.12 on n18.

S10.4.4.4.12: This state input [segBloodType, FinalRh] and output [segBloodType, FinalRh] a transition to on S13 on n19.

510.4.4.4.13: This is the end of the transitions of this sub-state and it output [segBlood-Type, FinalRh] the final ABO, Rh to S10.4.4.4.

FIG. 23 shows the composite state S13. This state checks the transfusion request prior to starting the transfusion. This section describes the transfusion request process.

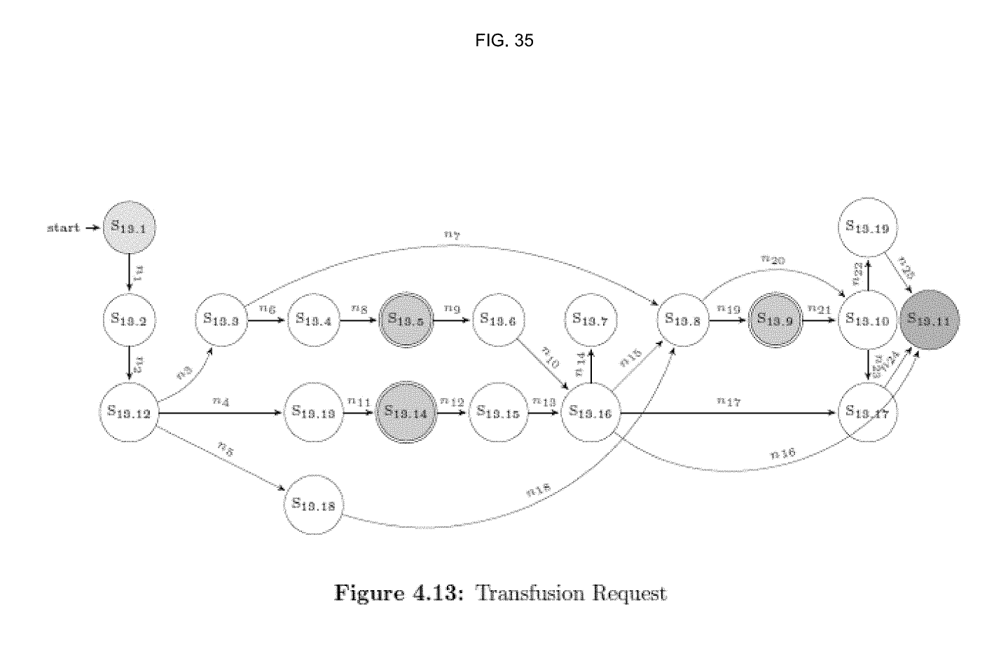

FIG. 35 illustrates transfusion request workflow modelled as state transitions between so-called states of the blood supply chain.

FIG. 35 shows the decomposition of state S13: is a composite state consists of states S13.1 to S13.11 used to model transfusion request.

S13.1 S13.1: S13.2 and S13.2 are empty states. On start of S13 it triggers this state to transition to S13.3.

S13.3: This state, the user enters [ElifeSaving, epriority, consent, verifiedby, verified-Date, ptFname, ptLastName, ptNo, ptAge, ptSex, pt availablesample, DateRequired, TimeRequired, RequestingDoc, DocCode, ReqRBC, ReqPLT, ReqFFP, ReqPRBC, ReqCRYO, PTsampleDate, groupSave, Crossmatch, PTbloodgroup,]. Based on trigger t270 to t278 a transition to S13.4 on n6 to check patient blood group or S13.8 on n7 to choose a unit.

S13.4: This state, the user enters [ABOrhHist, ptSampleNo, needPTsample]. A transition to composite state S13.5 on n8.

S13.5: This is a composite state to check the patient sample for the blood group. S13.5 decomposes into S13.5.1 to S13.5.13. On completion a transition to S13.6 on n9.

S13.6: This is an empty state. On start a transition to S13.16 on n10.

S13.7: This state, the user enters [BASneg]. A transition to S13.8 if specified to crossmatch or S13.17 if did not specify crossmatch.

S13.8: This state output [UnitNo, abo, rh]. This state interacts with the database, allowing the user to pick a unit for crossmatching. Upon completion a transition to S13.9.

S13.9: This is a composite state that decomposes into S13.9.1 to S13.9.7. Upon completion it transition to S13.10.

S13.10: This state is an empty state. A transition to S13.19 if the unit is required immediately or S13.17 if the unit or group and save are required to hold the unit or sample testing.

S13.13: This state the user enter [needPTsample] if another sample has been received. S transition to S13.14.

S13.14: This is a composite state. S13.14 decomposes into S13.14.1 to S13.14.13 to identify the new patient sample. S13.14 transition to S13.16.

513.15: This is an empty state that automatically transitions to state S13.16 on start.

S13.16: This state compare results from patient samples or patient blood group history. S13.16 transitions to state S13.8 if it is a life saving situation with no patient sample. S13.16 transition into state S13.7 for regulation transfusion with patient sample available. Non matching blood group with previous patient sample transitions to S13.11

S13.18: In this state the user is transitioned to state S13.8 in order to choose unit.

S13.11: This is an empty state that ends the transitions for this sub-state.

FIG. 35 shows the composite state S13.9 in pink. This state checks the selected unit compatibility with the patient blood. This section describes the crossmatch process.

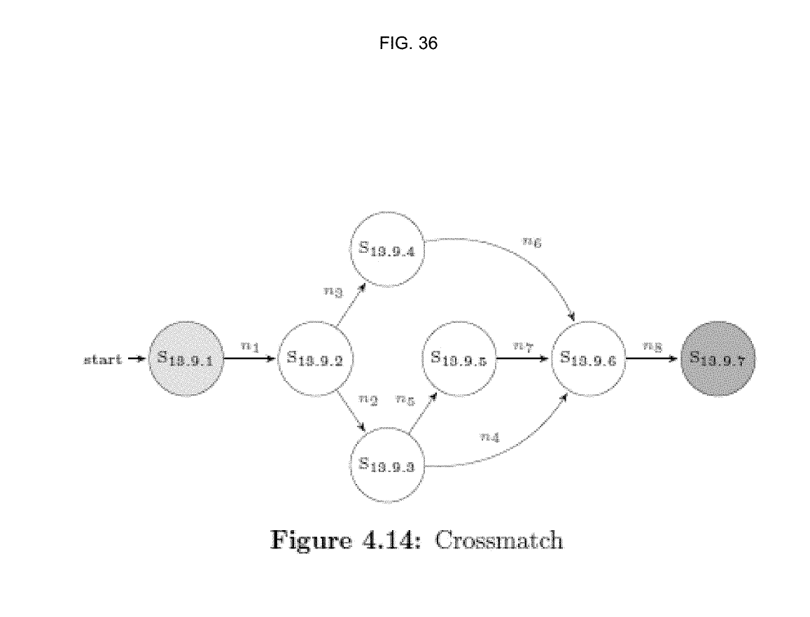

FIG. 36 illustrates cross-match workflow modelled as state transitions between so-called states of the blood supply chain.

FIG. 36 shows the decomposition of state S13.9: is a composite state consists of states S13.9.1 through S13.9.7 used to model crossmatch testing.

S13.9.1: This state input [PTbloodGroup, ptRHresult, ABSneg, ABO, Rh, UnitNo] but is triggered on start to transition into state S13.9.2.

S13.9.2: This state input [PTbloodGroup, ptRHresult, ABSneg, ABO, Rh, UnitNo]. Patients with Antibody screening negative are transitioned to state S13.9.3. If not S13.9.2 transitions into state S13.9.4 for serological crossmatching.

S13.9.3: This state checks if the selected unit and patient blood group are compatible. If compatible, S13.9.3 transition into state S13.9.5

S13.9.4: This state the user enters [UnitVerifiedMatchXM] if the unit is a match after performing the manual crossmatch. Then S13.9.4 transitions into state S13.9.6.

S13.9.5: This state input [UnitVerifiedMatchXM] to reverify if the crossmatch is a match. On completion S13.9.5 transitions into state S13.9.6.

S13.9.6: This is an empty state that transitions into state S13.9.7 on start.

S13.9.7: This is the final state for this sub-states. On trigger, S13.9.7 transitions to the higher level state S13.9.

FIG. 23 shows the composite state S14 shown in pink. This state checks and monitor the patient for transfusion. This section describes the patient transfusion process.

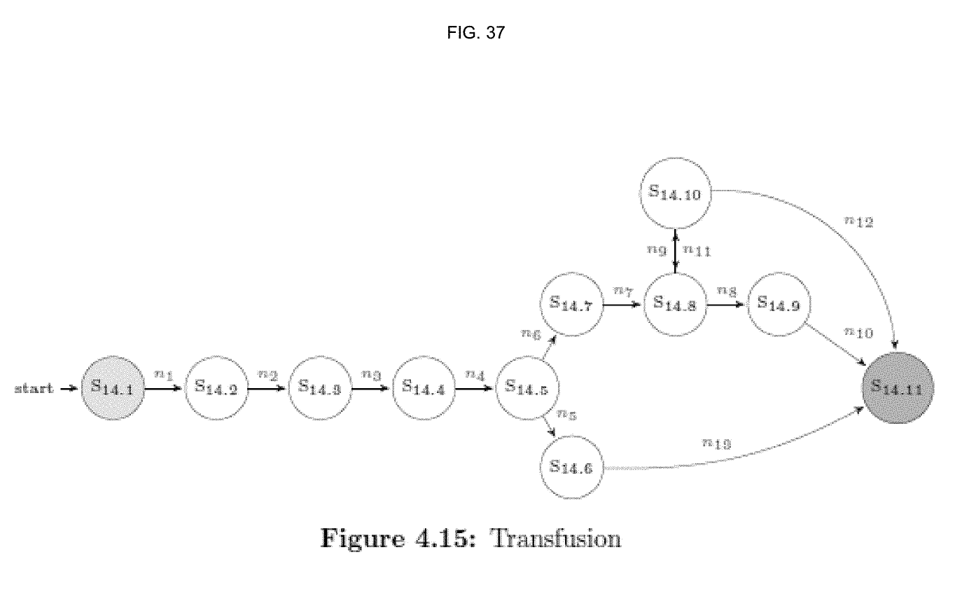

FIG. 37 illustrates transfusion workflow modelled as state transitions between so-called states of the blood supply chain.

FIG. 37 shows the decomposition of state S14: is a composite state consists of states S14.1 through S14.11 used to model transfusion.

S14.1 and S14.2: These are empty states. The decomposition of S14 start S14.1. S14.1 transitions to S14.2. Followed by another transition to S14.3.

S14.3: This state, checks the patient identity. The user enters [patientWristband, HospitalCode, nameCheck] as booleans. On completion a transition to S14.4.

S14.4: This is an empty state. On start it transitions to S14.5.

S14.5: This is a condition state. The user enters to “Stop Transfusion” or “Begin Transfusion”. This result in a transition into state S14.6 or S14.7.

S14.6: This state is an empty state, as the transfusion stops. On completion a transition into state S14.11.

S14.7: This state is an empty state, as the transfusion starts. On completion, S14.7 transition into state S14.8.

S14.8: This state checks the patient vitals [CheckVitals]. A boolean to verify checking patient vitals allowing a transition into state S14.10. Otherwise, S14.8 transitions into state S14.9 to end the transfusion if any transfusion reactions occurs.

S14.9: This is an empty state to end of the transfusion and stop it. On start, S14.9 transitions to state S14.10.

S14.10: This state checks if the patient status for no transfusion reactions. The user enters [Pass] boolean if the patient has no transfusion reaction. On trigger, a transition to state S14.8 to recheck the patient vitals. Otherwise, S14.10 transitions into state S14.9 to end the transfusion.

S14.11: This is the end state of this sub-state. S14.11 transitions into the higher-level state S14.

FIG. 35 shows the composite state S13.5. This state checks the patient blood at the laboratory for blood grouping test. This section describes the patient blood grouping process.

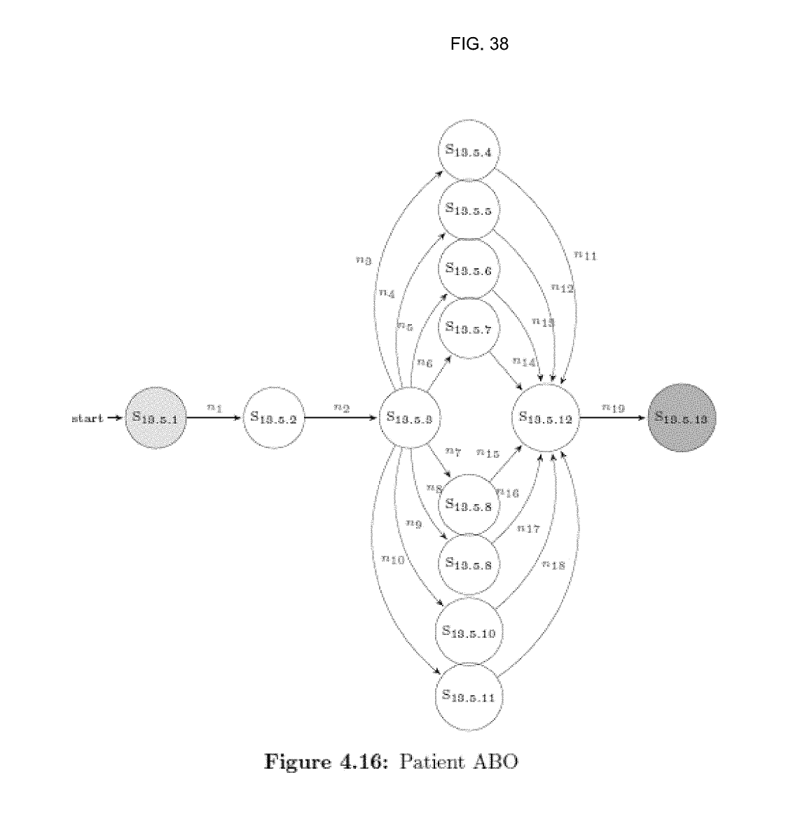

FIG. 38 illustrates patient ABO workflow modelled as state transitions between so-called states of the blood supply chain.

FIG. 38 shows the decomposition of state S13.5: is a composite state consists of states S13.5.1 through S13.5.13 used to model patient ABO testing.

S13.5.1: This is an empty state. It automatically transitions on start to S13.5.2.

S13.5.2: This is an empty state that starts ABO from sample and transitions to S13.5.3 on start.

S13.5.3: This state, the user enters [samAntiA, samAntiB, sam1anti1AB, samD6n, samCTL, samA1, samBCell] into the system. On trigger of t310 to t321 a transition to S13.5.4 on n3 or S13.5.5 on n4 or S13.5.6 on n5 or S13.5.7 on n6 or S13.5.8 on n7 or S13.5.9 on n8 or S13.5.10 on n9 or S13.5.11 on n10.

S13.5.4: On start the system input [samBloodABO, rhLocal] and output [samBlood-ABO, rhLocal]. To interpret the type of blood Type A POS. A transition to S13.5.12 on n11.

S13.5.5: On start the system input [samBloodABO, rhLocal] and output [samBlood-ABO, rhLocal]. To interpret the type of blood Type A NEG. A transition to S13.5.12 on n12.

S13.5.6: On start the system input [samBloodABO, rhLocal] and output [samBlood-ABO, rhLocal]. To interpret the type of blood Type B POS. A transition to S13.5.12 on n13.

S13.5.7: On start the system input [samBloodABO, rhLocal] and output [samBlood-ABO, rhLocal]. To interpret the type of blood Type B NEG. A transition to S13.5.12 on n14.

S13.5.8: On start the system input [samBloodABO, rhLocal] and output [samBlood-ABO, rhLocal]. To interpret the type of blood Type AB POS. A transition to S13.5.12 on n15.

S13.5.9: On start the system input [samBloodABO, rhLocal] and output [samBlood-ABO, rhLocal]. To interpret the type of blood Type AB NEG. A transition to S13.5.12 on n16.

S13.5.10: On start the system input [samBloodABO, rhLocal] and output [samBlood-ABO, rhLocal]. To interpret the type of blood Type 0 POS. A transition to S13.5.12 on n17

S13.5.11: On start the system input [samBloodABO, rhLocal] and output [samBlood-ABO, rhLocal]. To interpret the type of blood Type 0 NEG. A transition to S13.5.12 on n18.

S13.5.12: This state input [samBloodType, FinalRh] and output [samBloodType, FinalRh] transitions to S12.5.12 on n19.

S13.5.13: This is the end of the transitions of this sub-state and it output [samBlood-Type, FinalRh] the final ABO, Rh to S13.5

FIG. 4.17: Post Transfusion

FIG. 23 shows the composite state S15. This state monitors the patient after transfused with blood. This section describes the post transfusion process.

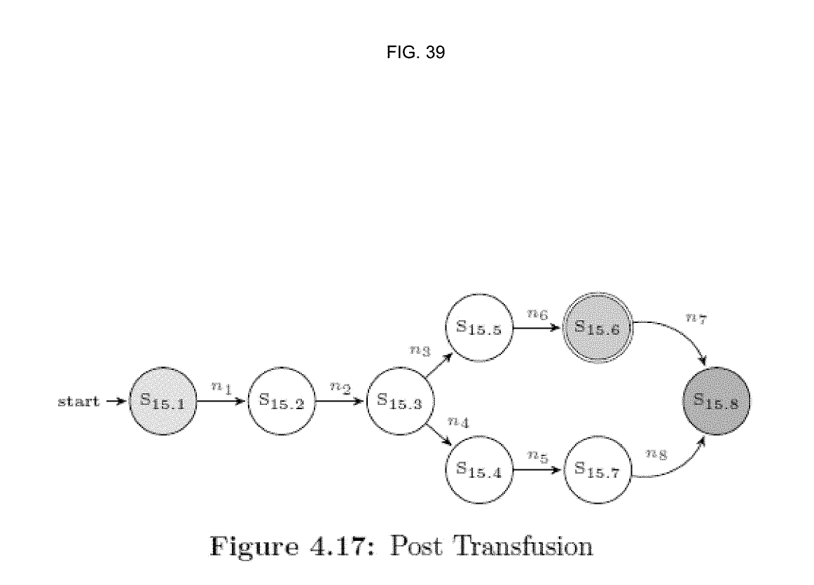

FIG. 39 illustrates post transfusion workflow modelled as state transitions between so-called states of the blood supply chain.

FIG. 39 shows the decomposition of state S15: is a composite state consists of states S15.1 to S15.7 used to model post transfusion.

S15.1: This is the start state. On start of S15 S15.1 transition to S15.1. This state is empty so on start this state transitions to S15.2.

S15.2: This state, the user enters boolean for [symptomsOrsigns] to show if the patient is showing any symptoms. On completion a transition to S15.3.

S15.3: This is a condition state. On trigger of on t309, the user chooses a transition to state S15.4 on n3 patient has no symptoms of transfusion reaction or S15.5 on n4 if the patient has symptoms of transfusion reaction.

S15.4: This is an empty state as the user is required to provide the patient with information documents prior to releasing the patient. On start of this state an automatic transition to S15.7 on n5.

S15.5: This is an empty state. On start a transition to S15.6 on n6 for patients with transfusion reaction.

S15.6: This is a composite state that decomposes into S15.6.1 to S15.6.7. On completion a transition to S15.8 on n7.

S15.7: This is an empty state where the patient is released. On start a transition to S15.8 on n8.

S15.8: This is an empty state that transitions to S15 when triggered.

FIG. 39 shows the composite state S15.6. This state identifies the type of adverse reaction the patient is experiencing. This section describes the transfusion adverse reaction process.

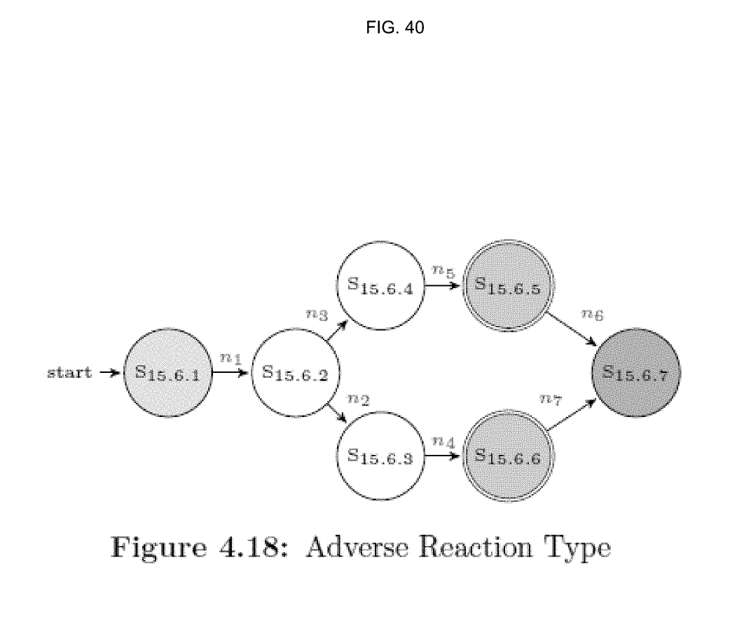

FIG. 40 illustrates adverse reaction type workflow modelled as state transitions between so-called states of the blood supply chain.

FIG. 40 shows the decomposition of state S15.6. State S15.6 is a composite state consisting of states S15.6.1 to S15.6.7 used to model transfusion adverse reaction type.

S15.6.1: This is an empty state. On start of S15.6 a transition to S15.6.1. S15.6.1 automatic transition to state S15.6.2 on input n1.

S15.6.2: This state checks if the symptoms occurred prior to 24 hours or after 24 hours. The user enters a boolean [moreThan24 hrs]. Symptoms occurring prior to the 24 hours after transfusion transition to S15.6.3. Else a transition to state S15.6.4. On trigger t294, S15.6.4 transitions to state S15.6.3 on trigger n2 or transition to state S15.6.4 on trigger n3.

S15.6.3: This state represents transfusion reactions that are prior to 24 hour period. The user is required to enter a boolean for any symptoms [chills, fever, hemoglobinuria, hypotension, renalFailureWithOliguria, DIC, backpain, painAndinfusionVein, anxiety, headache, vomiting, urticaria, pruritis, flushing, bonchospasm, localEdema, hypoxemia, respiratoryFailure, bilateralPulmonaryEdema, orthopnea, cough, dyspnea, paresthesia, tetany, arrhythmia, cardiacArrhythmia, posABS]. On completion, transition to state S15.6.6 on trigger n4.

S15.6.4: This state represents delayed transfusion reaction. The user enters a boolean for the patient symptoms [fever, plt Refratoriness, delayedHemolyticReaction, HDFN, decreasingHgb, newPOSANS, mildJanundice, Erythroderma, maculopapularRash, anorexia, diarrhea, hepatitis, pancytopenia, thrombocytopenicPurpura, bleeding, diabetes, cirrhosis, cardiomyopathy]. On completion, transition into state S15.6.5 on trigger n5.

S15.6.5: This is a composite state taking inputs [fever, plt Refratoriness, delayed-HemolyticReaction, HDFN, decreasingHgb, newPOSANS, mildJanundice, Erythroderma, maculopapularRash, anorexia, diarrhea, hepatitis, pancytopenia, thrombocytopenicPurpura, bleeding, diabetes, cirrhosis, cardiomyopathy]. The state identifies the type of delayed transfusion reaction the patient is having. On completion, transition to S15.6.7 on trigger n6.

S15.6.6: This is a composite state to identify the type of acute transfusion reaction the patient is having. On completion transition to state S15.6.7 on trigger n7.

S15.6.7: This is the end state. On completion a transition to S15.6.

FIG. 40 shows the composite state S15.6.6 in pink. This state identifies the type of acute adverse reaction the patient is experiencing. This section describes the transfusion acute adverse reaction process.

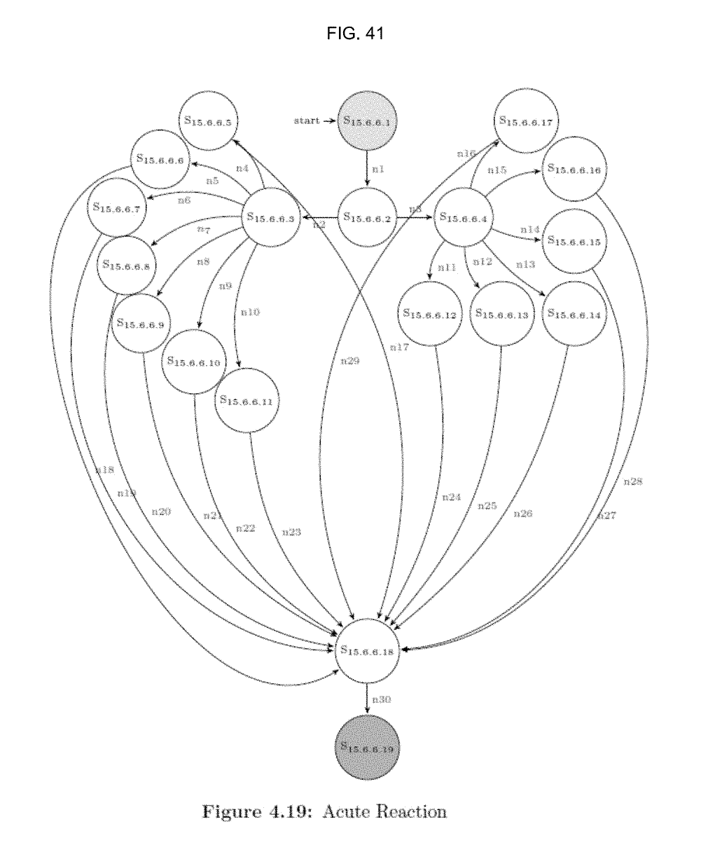

FIG. 41 illustrates acute reaction workflow modelled as state transitions between so-called states of the blood supply chain.

FIG. 41 shows the decomposition of state S15.6.6: is a composite state consists of states S15.6.6.1 to S15.6.6.19 used to model transfusion acute adverse reaction type.

S15.6.6.1: This is an empty state. On start of S15.6.6 a transition to S15.6.6.1.

S15.6.6.2: This state, the user is required to enter the patient symptoms as Boolean [chills, fever, hemoglobinuria, hypotension, renalfailurewitholig, DIC, backpain, pain and infusionvein, anxiety, headache, vomiting, urticaria, pruritis, flushing, bonchospasm, local edema, hypoxemia, respiratory failure, bilateral pulmonary EEdema, dyspnea, orthopnea, cough, cyanosis, paresthesia, tetany, arrhythmia, cardiacarrhythmia, pos-ABS]. Based on trigger of (on t322, t323, t324, t325, t326, t327, t328, t329, t330, t331, t332, t333, t336, t337, t322, t338, t339, t340) the entered data a transition to 515.6.6.3 on n2 nonimmunologic or S15.6.6.4 on trigger n3 immunologic.

S15.6.6.3: This state is an empty state that transitions to S15.6.6.5 on n4 or S15.6.6.6 on n5 or S15.6.6.7 on n6 or S15.6.6.8 on n7 or S15.6.6.9 on n8 or S15.6.6.10 on n9 or S15.6.6.11 on n10 for all nonimmunologic reactions.

S15.6.6.4: This state is an empty state that transitions to S15.6.6.12 on n11 or S15.6.6.13 on n12 or S15.6.6.14 on n13 or S15.6.6.15 on n14 or S15.6.6.16 on n15 or S15.6.6.17 on n16 for all immunologic reactions.

S15.6.6.5: This is an empty state that represents Air embalus reaction. This state can be expanded to show treatment and other details. The system automatic transitions into S15.6.6.18 on n17 on start.

S15.6.6.6: This is an empty state that represents Nonimmune hemolysis reaction. This state can be expanded to show treatment and other details. An automatic transition to S15.6.6.18 on n18 on start.

S15.6.6.7: This is an empty state that represents Transfusion associated circulatory overload reaction. This state can be expanded to show treatment and other details.

An automatically transition into state S15.6.6.18 on trigger n19 on start.

S15.6.6.8: This is an empty state that represents Hypotension associated with ACE inhibition reaction. This state can be expanded to show treatment and other details that automatically transition to state S15.6.6.18 on trigger n20 on start.

S15.6.6.9: This is an empty state that represents Transfusion associated sepsis reaction. This state can be expanded to show treatment and other details, automatically transition to state S15.6.6.18 on trigger n21 on start.

S15.6.6.10: This is an empty state that represents Hypocalcemia reaction. This state can be expanded to show treatment and other details, automatic transition to S15.6.6.18 on trigger n22 at start.

S15.6.6.11: This is an empty state that represents Hypothermia reaction. This state can be expanded to show treatment and other details. An automatic transition to S15.6.6.18 on n23 on start.

S15.6.6.12: This is an empty state that represents Alloimmunization RBC antigens reaction. This state can be expanded to show treatment and other details. An automatic transition to S15.6.6.18 on n24 on start.

S15.6.6.13: This is an empty state that represents Transfusion-related acute lung injury reaction. This state can be expanded to show treatment and other details. An automatic transition to S15.6.6.18 on n25 on start.

S15.6.6.14: This is an empty state that represents Anaphylactic reaction. This state can be expanded to show treatment and other details. An automatic transition to S15.6.6.18 on n26 on start.

S15.6.6.15: This is an empty state that represents Urticarial reaction. This state can be expanded to show treatment and other details. An automatic transition to S15.6.6.18 on n27 on start.

S15.6.6.16: This is an empty state that represents Febrile and nonhemoltic reaction. This state can be expanded to show treatment and other details. An automatic transition to S15.6.6.18 on n28 on start.

S15.6.6.17: This is an empty state that represents Hemolytic reaction. This state can be expanded to show treatment and other details. An automatic transition to S15.6.6.18 on n29 on start.

S15.6.6.18: This is an empty state. On start a transition on n30 to S15.6.6.19.

S15.6.6.19: This is an empty state and the last state of this sub-state transitions. On start a transition to S15.6.6.

FIG. 40 shows the composite state S15.6.5 in pink. This state identifies the type of delayed adverse reaction the patient is experiencing. This section describes the transfusion delayed adverse reaction process.

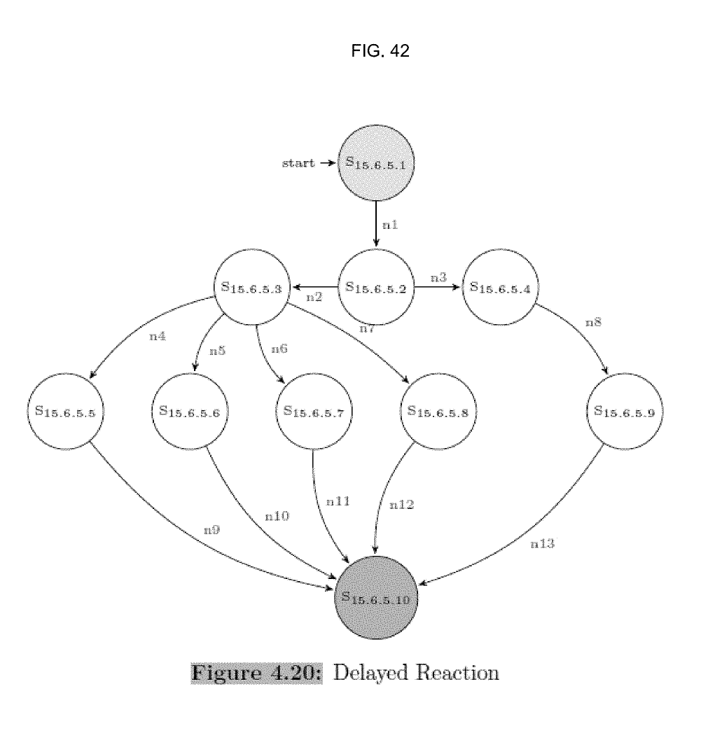

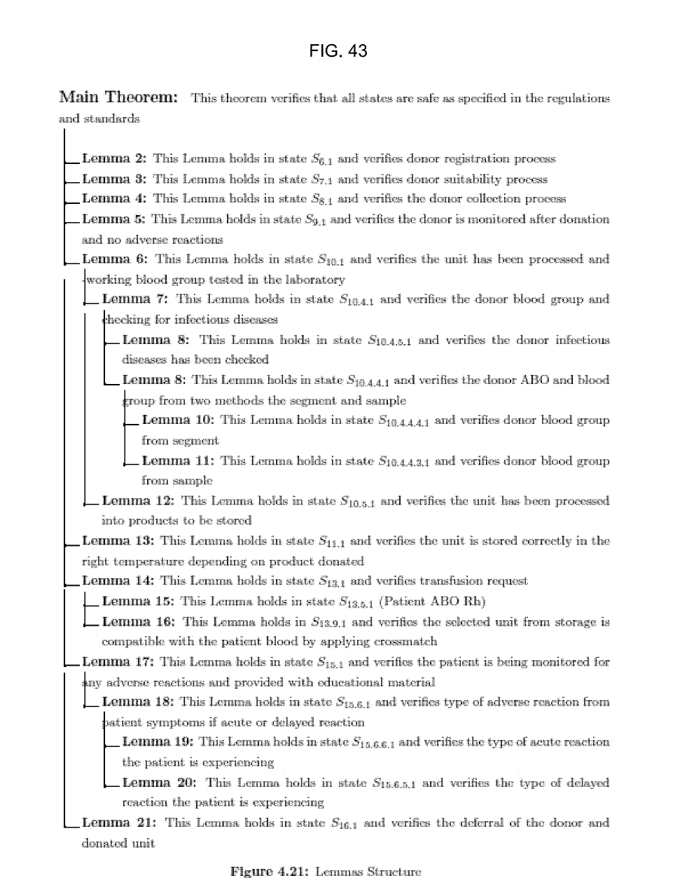

FIG. 42 illustrates delayed reaction workflow modelled as state transitions between so-called states of the blood supply chain.

FIG. 42 shows the decomposition of state S15.6.6: is a composite state consists of states S15.6.5.1 to S15.6.5.10 used to model transfusion delayed adverse reaction type.

S15.6.5.1: This is an empty state. On start of S15.6.5 a transition to S15.6.5.1.

S15.6.5.2: This state, the user is required to enter the patient symptoms as Boolean [fever, plt Refratoriness, delayedHemolyticReaction, HDFN, decreasingHgb, newPOSANS, mildJanundice, Erythroderma, maculopapularRash, anorexia, diarrhea, hepatitis, pancytopenia, thrombocytopenicPurpura, bleeding, diabetes, cirrhosis, cardiomyopathy]. Based on the entered data a transition to S15.6.5.3 nonimmunologic or S15.6.5.4 immunologic.

S15.6.5.3: This state is an empty state that transitions to S15.6.5.5 on n4 or S15.6.5.6 on n5 or S15.6.5.7 on n6 or S15.6.5.8 on n7 for all Delayed immunologic reactions.

S15.6.5.4: This state is an empty state that transitions to S15.6.5.9 on n8 if the entered symptoms are diabetes, cirrhosis, cardiomyopathy for Delayed nonimmunologic reactions.

S15.6.5.5: This is an empty state that represent Alloimmunization HLA antigens reaction. This state can be expanded to show treatment and other details. An automatic transition to S15.6.5.18 on n9 on start.

S15.6.5.6: This is an empty state that represent Hemolytic reaction. This state can be expanded to show treatment and other details. An automatic transition to S15.6.5.18 on n10 on start.

S15.6.5.7: This is an empty state that represent Graft vs host disease reaction. This state can be expanded to show treatment and other details. An automatic transition to S15.6.5.18 on n11 on start.

S15.6.5.8: This is an empty state that represent Post transfusion purpura reaction. This state can be expanded to show treatment and other details. An automatic transition to S15.6.5.19 on n12 on start.

S15.6.5.9: This is an empty state that represent Iron overload reaction. This state can be expanded to show treatment and other details. An automatic transition to S15.6.5.18 on n13 on start.

S15.6.5.10: This is an empty state that represent the end of this sub-state. A transition to S15.6.5 to complete this sub-state.

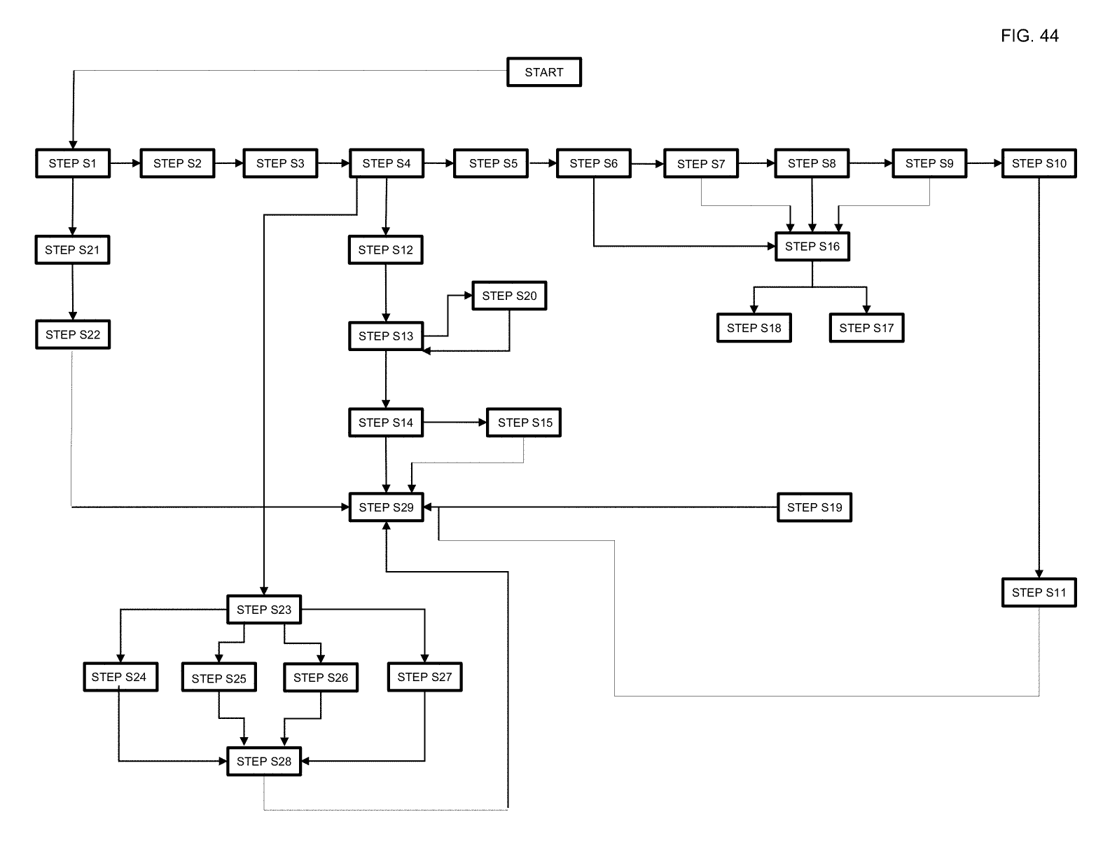

FIG. 44 illustrates a flow chart of the modelled workflow illustrated in FIG. 23.

Referring to FIG. 44, the operation steps of the method according to an embodiment of the present inventive concept to verify and validate blood safety work flows correspond to the S# referred throughout. For instance, Step S1 refers to S1 illustrated in FIG. 23. The steps are preformed according to the temporal logic formulas and whether or not the specified regulation or standard has been satisfied. However, the present general inventive concept is not limited thereto.

The present general inventive concept includes a method of using temporal logic technique to verify and validate blood safety workflows which includes creating a model workflow of blood supply chain process, wherein humans and machines are included as workflow constructs where involved, translating regulations governing blood safety into a formula, wherein components satisfying the formula correspond to satisfying the translated regulation, combining the translated regulations with the created blood supply chain model workflow and validating that all translated regulations have been satisfied using a theorem prover.

The blood safety regulations may be translated into a plurality of statements in a temporal logic formula. The model workflow includes selecting a country, determining regulations based on the selected country, checking donor requirements for registration, verifying safety of the donor requirements found in a database, allowing donor to register and donate, checking vitals of donor to verify suitability and safety of donor's blood, collecting blood from the donor, checking application of safety controls to collection of donor's blood, determining whether donor has adverse reactions, checking collected blood for infectious diseases and donor blood grouping, verifying requirements of blood storage, storing collected blood based on verified blood storage requirements, verifying donor information and performing serology testing on the collected blood. The verifying requirements of blood storage further includes verifying correct labelling.

The present general inventive concept can also be embodied as computer-readable codes on a computer-readable medium. The computer-readable medium can include a computer-readable recording medium and a computer-readable transmission medium. The computer-readable recording medium is any data storage device that can store data as a program which can be thereafter read by a computer system. Examples of the computer-readable recording medium include read-only memory (ROM), random-access memory (RAM), CD-ROMs, DVDs, magnetic tapes, floppy disks, and optical data storage devices. The computer-readable recording medium can also be distributed over network coupled computer systems so that the computer-readable code is stored and executed in a distributed fashion. The computer-readable transmission medium can transmit carrier waves or signals (e.g., wired or wireless data transmission through the Internet).

Although a few exemplary embodiments of the present general inventive concept have been illustrated and described, it will be appreciated by those skilled in the art that changes may be made in these exemplary embodiments without departing from the principles and spirit of the general inventive concept, the scope of which is defined in the appended claims and their equivalents.

What is claimed is:

1. A method of using temporal logic technique to verify and validate blood safety workflows, the method comprising:
creating a model workflow of blood supply chain process, wherein humans and machines are included as workflow constructs where involved;
translating regulations governing blood safety into statements in temporal logic formula, wherein components satisfying a temporal logic formula correspond to satisfying the translated regulation;
combining the translated regulations with the created blood supply chain model workflow; and
validating that all translated regulations have been satisfied using a theorem prover.

2. The method of claim 1, further comprising a model checker to prove that the created model workflow satisfies all of the regulations governing blood safety.

3. The method of claim 1, further comprising using formal workflow specification and execution environment and language in Yet Another Workflow Model (YAWL) to create the model workflow of the blood supply chain process.

4. The method of claim 3, further comprising using YAWL to check that the created model workflow satisfies reachability, structure, and soundness.

5. The method of claim 1, wherein the created model workflow of the blood supply chain process includes registering a donor, conducting a physical exam, conducting an interview, determining eligibility, creating labels, drawing blood, determining post donation status, and transfusing blood.

6. The method of claim 1, wherein the validating of the translated regulations occurs automatically.

7. A method of using temporal logic technique to verify and validate blood safety workflows, the method comprising:
creating a model workflow of blood supply chain process, wherein humans and machines are included as workflow constructs where involved;
translating regulations governing blood safety into a formula, wherein components satisfying the formula correspond to satisfying the translated regulation;
combining the translated regulations with the created blood supply chain model workflow; and
validating that all translated regulations have been satisfied using a theorem prover.

8. The method of claim 7, wherein the blood safety regulations are translated into statements in a temporal logic formula.

9. The method of claim 8, the model workflow comprises:
selecting a country;
determining regulations based on the selected country:
checking donor requirements for registration;
verifying safety of the donor requirements;
allowing donor to register and donate;
checking vitals of donor to verify suitability and safety of donor's blood;
collecting blood from donor;
checking application of safety controls to collection of donor's blood;
determining whether donor has adverse reactions;
checking collected blood for infectious diseases and donor blood grouping;
verifying requirements of blood storage;
storing collected blood based on verified blood storage requirements;
verifying donor information; and
performing serology testing on the collected blood.

10. The method of claim 9, wherein the verifying requirements of blood storage includes verifying correct labelling.
